SEQUENCE LISTING (134 sequences)

SEQ ID NO: 1 - DNA, 600 bp, Aequorea victoria
  CDS (1)..(600)
atgctttatgatgttcctgattatgctagcctcaaacttacatcagacttcgacaacccaagatggattggacgacacaagcatatgttcaatttccttgatgtcaaccacaatggaaaaatctctcttgacgagatggtctacaaggcatctgatattgtcatcaataaccttggagcaacacctgagcaagccaaacgacacaaagatgctgtagaagccttcttcggaggagctggaatgaaatatggtgtggaaactgattggcctgcatatattgaaggatggaaaaaattggctactgatgaattggagaaatacgccaaaaacgaaccaacgctcatccgtatatggggtgatgctttgtttgatatcgttgacaaagatcaaaatggagccattacactggatgaatggaaagcatacaccaaagctgctggtatcatccaatcatcagaagattgcgaggaaacattcagagtgtgcgatattgatgaaagtggacaactcgatgttgatgagatgacaagacaacatttaggattttggtacaccatggatcctgcttgcgaaaagctctacggtggagctgtcccctaa

SEQ ID NO: 2 - protein, 199 aa, Aequorea victoria
MLYDVPDYASLKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGQLDVDEMTRQHLGFWYTMDPACEKLYGGAVP

SEQ ID NO: 3 - DNA, 600 bp, artificial sequence
  sequence derived by mutation of aequorin
atgctttatgatgttcctgattatgctagcctcaaacttacatcagacttcgacaacccaagatggattggacgacacaggcatatgttcaatttccttgatgtcaaccacaatggaaaaatctctcttgacgagatggtctacaaggcatctgatattgtcatcaataaccttggagcaacacctgagcaagccaaacgacacaaagatgctgtagaagccttcttcggaggagctggaatgaaatatggtgtggaaactgattggcctgcatatattgaaggatggaaaaaattggctactgatgaattggagaaatacgccaaaaacgaaccaacgctcatccgtatatggggtgatgctttgtttgatatcgttgacaaagatcaaaatggagccattacactggatgaatggaaagcatacaccaaagctgctggtatcatccaatcatcagaagattgcgaggaaacattcagagtgtgcgatattgatgaaagtggacaactcgatgttgatgagatgacaagacaacatttaggattttggtacaccatggatcctgcttgcgaaaagctctacggtggagctgtcccctaa

SEQ ID NO: 4 - protein, 199 aa, artificial sequence
  sequence derived by mutation of aequorin
MLYDVPDYASLKLTSDFDNPRWIGRHRHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGQLDVDEMTRQHLGFWYTMDPACEKLYGGAVP

SEQ ID NO: 5 - DNA, 600 bp, artificial sequence
  sequence derived by mutation of aequorin
atgctttatgatgttcctgattatgctagcctcaaacttacatcagacttcgacaacccaagatggattggacgacacaagcatatgttcaatttccttgatgtcaaccacaatggaaaaatctctcttgacgagatggtctacaaggcatctgatattgtcatcaataaccttggagcaacacctgagcaagccaaacgacacaaagatgctgtagaagccttcttcggaggagctggaatgaaatatggtgtggaaactgattggcctgcatatattgaaggatggaaaaaattggctactgatgaattggagaaatacgccaaaaacgaaccaacgctcatccgtatatggggtgatgctttgtttgatatcgttgacaaagatcaaaatggagccattacactggatgaatggaaagcatacaccaaagctgctggtatcatccaatcatcagaagattgcgaggaaacattcagagtgtgcgatattgatgaaagtggacaactcgatgttgatgagatgacaagacgacatttaggattttggtacaccatggatcctgcttgcgaaaagctctacggtggagctgtcccctaa

SEQ ID NO: 6 - protein, 199 aa, artificial sequence
  sequence derived by mutation of aequorin
MLYDVPDYASLKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGQLDVDEMTRRHLGFWYTMDPACEKLYGGAVP

SEQ ID NO: 7 - DNA, 600 bp, artificial sequence
  sequence derived by mutation of aequorin
atgctttatgatgttcctgattatgctagcctcaaacttacatcagacttcgacaacccaagatggattggacgacacaagcatatgttcaatttccttgatgtcaaccacaatggaaaaatctctcttgacgagatggtctacaaggcatctgatattgtcatcaataaccttggagcaacacctgagcaagccaaacgacacaaagatgctgtagaagccttcttcggaggagctggaatgaaatatggtgtggaaactgattggcctgcatatattgaaggatggaaaaaattggctactgatgaattggagaaatacgccaaaaacgaaccaacgctcatccgtatatggggtgatgctttgtttgatatcgttgacaaagatcaaaatggagccattacactggatgaatggaaagcatacaccaaagctgctggtatcatccaatcatcagaagattgcgaggaaacattcagagtgtgcgatattgatgaaagtggacaactcgatgttgatgagatgacaagacaacatataggattttggtacaccatggatcctgcttgcgaaaagctctacggtggagctgtcccctaa

SEQ ID NO: 8 - protein, 199 aa, artificial sequence
  sequence derived by mutation of aequorin
MLYDVPDYASLKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGQLDVDEMTRQHIGFWYTMDPACEKLYGGAVP

SEQ ID NO: 9 - DNA, 600 bp, artificial sequence
  sequence derived by mutation of aequorin
atgctttatgatgttcctgattatgctagcctcaaacttacatcagacttcgacaacccaagatggattggacgacacaggcatatgttcaatttccttgatgtcaaccacaatggaaaaatctctcttgacgagatggtctacaaggcatctgatattgtcatcaataaccttggagcaacacctgagcaagccaaacgacacaaagatgctgtagaagccttcttcggaggagctggaatgaaatatggtgtggaaactgattggcctgcatatattgaaggatggaaaaaattggctactgatgaattggagaaatacgccaaaaacgaaccaacgctcatccgtatatggggtgatgctttgtttgatatcgttgacaaagatcaaaatggagccattacactggatgaatggaaagcatacaccaaagctgctggtatcatccaatcatcagaagattgcgaggaaacattcagagtgtgcgatattgatgaaagtggacaactcgatgttgatgagatgacaagacgacatttaggattttggtacaccatggatcctgcttgcgaaaagctctacggtggagctgtcccctaa

SEQ ID NO: 10 - protein, 199 aa, artificial sequence
  sequence derived by mutation of aequorin
MLYDVPDYASLKLTSDFDNPRWIGRHRHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGQLDVDEMTRRHLGFWYTMDPACEKLYGGAVP

SEQ ID NO: 11 - DNA, 600 bp, artificial sequence
  sequence derived by mutation of aequorin
atgctttatgatgttcctgattatgctagcctcaaacttacatcagacttcgacaacccaagatggattggacgacacaggcatatgttcaatttccttgatgtcaaccacaatggaaaaatctctcttgacgagatggtctacaaggcatctgatattgtcatcaataaccttggagcaacacctgagcaagccaaacgacacaaagatgctgtagaagccttcttcggaggagctggaatgaaatatggtgtggaaactgattggcctgcatatattgaaggatggaaaaaattggctactgatgaattggagaaatacgccaaaaacgaaccaacgctcatccgtatatggggtgatgctttgtttgatatcgttgacaaagatcaaaatggagccattacactggatgaatggaaagcatacaccaaagctgctggtatcatccaatcatcagaagattgcgaggaaacattcagagtgtgcgatattgatgaaagtggacaactcgatgttgatgagatgacaagacaacatataggattttggtacaccatggatcctgcttgcgaaaagctctacggtggagctgtcccctaa

SEQ ID NO: 12 - protein, 199 aa, artificial sequence
  sequence derived by mutation of aequorin
MLYDVPDYASLKLTSDFDNPRWIGRHRHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGQLDVDEMTRQHIGFWYTMDPACEKLYGGAVP

SEQ ID NO: 13 - DNA, 600 bp, artificial sequence
  sequence derived by mutation of aequorin
atgctttatgatgttcctgattatgctagcctcaaacttacatcagacttcgacaacccaagatggattggacgacacaagcatatgttcaatttccttgatgtcaaccacaatggaaaaatctctcttgacgagatggtctacaaggcatctgatattgtcatcaataaccttggagcaacacctgagcaagccaaacgacacaaagatgctgtagaagccttcttcggaggagctggaatgaaatatggtgtggaaactgattggcctgcatatattgaaggatggaaaaaattggctactgatgaattggagaaatacgccaaaaacgaaccaacgctcatccgtatatggggtgatgctttgtttgatatcgttgacaaagatcaaaatggagccattacactggatgaatggaaagcatacaccaaagctgctggtatcatccaatcatcagaagattgcgaggaaacattcagagtgtgcgatattgatgaaagtggacaactcgatgttgatgagatgacaagacgacatataggattttggtacaccatggatcctgcttgcgaaaagctctacggtggagctgtcccctaa

SEQ ID NO: 14 - protein, 199 aa, artificial sequence
  sequence derived by mutation of aequorin
MLYDVPDYASLKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGQLDVDEMTRRHIGFWYTMDPACEKLYGGAVP

SEQ ID NO: 15 - DNA, 600 bp, artificial sequence
  sequence derived by mutation of aequorin
atgctttatgatgttcctgattatgctagcctcaaacttacatcagacttcgacaacccaagatggattggacgacacaggcatatgttcaatttccttgatgtcaaccacaatggaaaaatctctcttgacgagatggtctacaaggcatctgatattgtcatcaataaccttggagcaacacctgagcaagccaaacgacacaaagatgctgtagaagccttcttcggaggagctggaatgaaatatggtgtggaaactgattggcctgcatatattgaaggatggaaaaaattggctactgatgaattggagaaatacgccaaaaacgaaccaacgctcatccgtatatggggtgatgctttgtttgatatcgttgacaaagatcaaaatggagccattacactggatgaatggaaagcatacaccaaagctgctggtatcatccaatcatcagaagattgcgaggaaacattcagagtgtgcgatattgatgaaagtggacaactcgatgttgatgagatgacaagacgacatataggattttggtacaccatggatcctgcttgcgaaaagctctacggtggagctgtcccctaa

SEQ ID NO: 16 - protein, 199 aa, artificial sequence
  sequence derived by mutation of aequorin
MLYDVPDYASLKLTSDFDNPRWIGRHRHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGQLDVDEMTRRHIGFWYTMDPACEKLYGGAVP

SEQ ID NO: 17 - DNA, 597 bp, Clytia gregaria
  CDS (1)..(597)
atggctgacactgcatcaaaatacgccgtcaaactcagacccaacttcgacaacccaaaatgggtcaacagacacaaatttatgttcaactttttggacattaacggcgacggaaaaatcactttggatgaaatcgtctccaaagcttcggatgacatttgcgccaaacttggagcaacaccagaacagaccaaacgtcaccaggatgctgtcgaagctttcttcaaaaagattggtatggattatggtaaagaagtcgaattcccagcttttgttgatggatggaaagaactggccaattatgacttgaaactttggtctcaaaacaagaaatctttgatccgcgactggggagaagctgttttcgacatttttgacaaagacggaagtggctcaatcagtttggacgaatggaaggcttatggacgaatctctggaatctgctcatcagacgaagacgccgaaaagaccttcaaacattgcgatttggacaacagtggcaaacttgatgttgatgagatgaccagacaacatttgggattctggtacaccttggaccccaacgctgatggtctttacggcaattttgttccttaa

SEQ ID NO: 18 - protein, 198 aa, Clytia gregaria
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGKLDVDEMTRQHLGFWYTLDPNADGLYGNFVP

SEQ ID NO: 19 - DNA, 597 bp, artificial sequence
  sequence derived by mutation of clytin
atggctgacactgcatcaaaatacgccgtcaaactcagacccaacttcgacaacccaaaatgggtcaacagacacagatttatgttcaactttttggacattaacggcgacggaaaaatcactttggatgaaatcgtctccaaagcttcggatgacatttgcgccaaacttggagcaacaccagaacagaccaaacgtcaccaggatgctgtcgaagctttcttcaaaaagattggtatggattatggtaaagaagtcgaattcccagcttttgttgatggatggaaagaactggccaattatgacttgaaactttggtctcaaaacaagaaatctttgatccgcgactggggagaagctgttttcgacatttttgacaaagacggaagtggctcaatcagtttggacgaatggaaggcttatggacgaatctctggaatctgctcatcagacgaagacgccgaaaagaccttcaaacattgcgatttggacaacagtggcaaacttgatgttgatgagatgaccagacaacatttgggattctggtacaccttggaccccaacgctgatggtctttacggcaattttgttccttaa

SEQ ID NO: 20 - protein, 198 aa, artificial sequence
  sequence derived by mutation of clytin
MADTASKYAVKLRPNFDNPKWVNRHRFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGKLDVDEMTRQHLGFWYTLDPNADGLYGNFVP

SEQ ID NO: 21 - DNA, 597 bp, artificial sequence
  sequence derived by mutation of clytin
atggctgacactgcatcaaaatacgccgtcaaactcagacccaacttcgacaacccaaaatgggtcaacagacacaaatttatgttcaactttttggacattaacggcgacggaaaaatcactttggatgaaatcgtctccaaagcttcggatgacatttgcgccaaacttggagcaacaccagaacagaccaaacgtcaccaggatgctgtcgaagctttcttcaaaaagattggtatggattatggtaaagaagtcgaattcccagcttttgttgatggatggaaagaactggccaattatgacttgaaactttggtctcaaaacaagaaatctttgatccgcgactggggagaagctgttttcgacatttttgacaaagacggaagtggctcaatcagtttggacgaatggaaggcttatggacgaatctctggaatctgctcatcagacgaagacgccgaaaagaccttcaaacattgcgatttggacaacagtggcaaacttgatgttgatgagatgaccagacgacatttgggattctggtacaccttggaccccaacgctgatggtctttacggcaattttgttccttaa

SEQ ID NO: 22 - protein, 198 aa, artificial sequence
  sequence derived by mutation of clytin
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGKLDVDEMTRRHLGFWYTLDPNADGLYGNFVP

SEQ ID NO: 23 - DNA, 597 bp, artificial sequence
  sequence derived by mutation of clytin
atggctgacactgcatcaaaatacgccgtcaaactcagacccaacttcgacaacccaaaatgggtcaacagacacaaatttatgttcaactttttggacattaacggcgacggaaaaatcactttggatgaaatcgtctccaaagcttcggatgacatttgcgccaaacttggagcaacaccagaacagaccaaacgtcaccaggatgctgtcgaagctttcttcaaaaagattggtatggattatggtaaagaagtcgaattcccagcttttgttgatggatggaaagaactggccaattatgacttgaaactttggtctcaaaacaagaaatctttgatccgcgactggggagaagctgttttcgacatttttgacaaagacggaagtggctcaatcagtttggacgaatggaaggcttatggacgaatctctggaatctgctcatcagacgaagacgccgaaaagaccttcaaacattgcgatttggacaacagtggcaaacttgatgttgatgagatgaccagacaacatataggattctggtacaccttggaccccaacgctgatggtctttacggcaattttgttccttaa

SEQ ID NO: 24 - protein, 198 aa, artificial sequence
  sequence derived by mutation of clytin
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGKLDVDEMTRQHIGFWYTLDPNADGLYGNFVP

SEQ ID NO: 25 - DNA, 597 bp, artificial sequence
  sequence derived by mutation of clytin
atggctgacactgcatcaaaatacgccgtcaaactcagacccaacttcgacaacccaaaatgggtcaacagacacagatttatgttcaactttttggacattaacggcgacggaaaaatcactttggatgaaatcgtctccaaagcttcggatgacatttgcgccaaacttggagcaacaccagaacagaccaaacgtcaccaggatgctgtcgaagctttcttcaaaaagattggtatggattatggtaaagaagtcgaattcccagcttttgttgatggatggaaagaactggccaattatgacttgaaactttggtctcaaaacaagaaatctttgatccgcgactggggagaagctgttttcgacatttttgacaaagacggaagtggctcaatcagtttggacgaatggaaggcttatggacgaatctctggaatctgctcatcagacgaagacgccgaaaagaccttcaaacattgcgatttggacaacagtggcaaacttgatgttgatgagatgaccagacgacatttgggattctggtacaccttggaccccaacgctgatggtctttacggcaattttgttccttaa

SEQ ID NO: 26 - protein, 198 aa, artificial sequence
  sequence derived by mutation of clytin
MADTASKYAVKLRPNFDNPKWVNRHRFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGKLDVDEMTRRHLGFWYTLDPNADGLYGNFVP

SEQ ID NO: 27 - DNA, 597 bp, artificial sequence
  sequence derived by mutation of clytin
atggctgacactgcatcaaaatacgccgtcaaactcagacccaacttcgacaacccaaaatgggtcaacagacacagatttatgttcaactttttggacattaacggcgacggaaaaatcactttggatgaaatcgtctccaaagcttcggatgacatttgcgccaaacttggagcaacaccagaacagaccaaacgtcaccaggatgctgtcgaagctttcttcaaaaagattggtatggattatggtaaagaagtcgaattcccagcttttgttgatggatggaaagaactggccaattatgacttgaaactttggtctcaaaacaagaaatctttgatccgcgactggggagaagctgttttcgacatttttgacaaagacggaagtggctcaatcagtttggacgaatggaaggcttatggacgaatctctggaatctgctcatcagacgaagacgccgaaaagaccttcaaacattgcgatttggacaacagtggcaaacttgatgttgatgagatgaccagacaacatataggattctggtacaccttggaccccaacgctgatggtctttacggcaattttgttccttaa

SEQ ID NO: 28 - protein, 198 aa, artificial sequence
  sequence derived by mutation of clytin
MADTASKYAVKLRPNFDNPKWVNRHRFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGKLDVDEMTRQHIGFWYTLDPNADGLYGNFVP

SEQ ID NO: 29 - DNA, 597 bp, artificial sequence
  sequence derived by mutation of clytin
atggctgacactgcatcaaaatacgccgtcaaactcagacccaacttcgacaacccaaaatgggtcaacagacacaaatttatgttcaactttttggacattaacggcgacggaaaaatcactttggatgaaatcgtctccaaagcttcggatgacatttgcgccaaacttggagcaacaccagaacagaccaaacgtcaccaggatgctgtcgaagctttcttcaaaaagattggtatggattatggtaaagaagtcgaattcccagcttttgttgatggatggaaagaactggccaattatgacttgaaactttggtctcaaaacaagaaatctttgatccgcgactggggagaagctgttttcgacatttttgacaaagacggaagtggctcaatcagtttggacgaatggaaggcttatggacgaatctctggaatctgctcatcagacgaagacgccgaaaagaccttcaaacattgcgatttggacaacagtggcaaacttgatgttgatgagatgaccagacgacatataggattctggtacaccttggaccccaacgctgatggtctttacggcaattttgttccttaa

SEQ ID NO: 30 - protein, 198 aa, artificial sequence
  sequence derived by mutation of clytin
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGKLDVDEMTRRHIGFWYTLDPNADGLYGNFVP

SEQ ID NO: 31 - DNA, 597 bp, artificial sequence
  sequence derived by mutation of clytin
atggctgacactgcatcaaaatacgccgtcaaactcagacccaacttcgacaacccaaaatgggtcaacagacacagatttatgttcaactttttggacattaacggcgacggaaaaatcactttggatgaaatcgtctccaaagcttcggatgacatttgcgccaaacttggagcaacaccagaacagaccaaacgtcaccaggatgctgtcgaagctttcttcaaaaagattggtatggattatggtaaagaagtcgaattcccagcttttgttgatggatggaaagaactggccaattatgacttgaaactttggtctcaaaacaagaaatctttgatccgcgactggggagaagctgttttcgacatttttgacaaagacggaagtggctcaatcagtttggacgaatggaaggcttatggacgaatctctggaatctgctcatcagacgaagacgccgaaaagaccttcaaacattgcgatttggacaacagtggcaaacttgatgttgatgagatgaccagacgacatataggattctggtacaccttggaccccaacgctgatggtctttacggcaattttgttccttaa

SEQ ID NO: 32 - protein, 198 aa, artificial sequence
  sequence derived by mutation of clytin
MADTASKYAVKLRPNFDNPKWVNRHRFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGKLDVDEMTRRHIGFWYTLDPNADGLYGNFVP

SEQ ID NO: 33 - DNA, 597 bp, Mitrocoma cellularia
  CDS (1)..(597)
atgtcaatgggcagcagatacgcagtcaagcttacgactgactttgataatccaaaatggattgctcgacacaagcacatgttcaacttccttgacatcaattcaaatggccaaatcaatctgaatgaaatggtccataaggcttcaaacattatctgcaagaagcttggagcaacagaagaacaaaccaaacgtcatcaaaagtgtgtcgaagacttctttgggggagctggtttggaatatgacaaagataccacatggcctgagtacatcgaaggatggaagaggttggctaagactgaattggaaaggcattcaaagaatcaagtcacattgatccgattatggggtgatgctttgttcgacatcattgacaaagatagaaatggatcggtttcgttagacgaatggatccagtacactcattgtgctggcatccaacagtcacgtgggcaatgcgaagctacatttgcacattgcgatttagatggtgacggtaaacttgatgtggacgaaatgacaagacaacatttgggattttggtattcggtcgacccaacttgtgaaggactctacggtggtgctgtaccttattaa

SEQ ID NO: 34 - protein, 198 aa, Mitrocoma cellularia
MSMGSRYAVKLTTDFDNPKWIARHKHMFNFLDINSNGQINLNEMVHKASNIICKKLGATEEQTKRHQKCVEDFFGGAGLEYDKDTTWPEYIEGWKRLAKTELERHSKNQVTLIRLWGDALFDIIDKDRNGSVSLDEWIQYTHCAGIQQSRGQCEATFAHCDLDGDGKLDVDEMTRQHLGFWYSVDPTCEGLYGGAVPY

SEQ ID NO: 35 - DNA, 597 bp, artificial sequence
  sequence derived by mutation of mitrocomin
atgtcaatgggcagcagatacgcagtcaagcttacgactgactttgataatccaaaatggattgctcgacacaggcacatgttcaacttccttgacatcaattcaaatggccaaatcaatctgaatgaaatggtccataaggcttcaaacattatctgcaagaagcttggagcaacagaagaacaaaccaaacgtcatcaaaagtgtgtcgaagacttctttgggggagctggtttggaatatgacaaagataccacatggcctgagtacatcgaaggatggaagaggttggctaagactgaattggaaaggcattcaaagaatcaagtcacattgatccgattatggggtgatgctttgttcgacatcattgacaaagatagaaatggatcggtttcgttagacgaatggatccagtacactcattgtgctggcatccaacagtcacgtgggcaatgcgaagctacatttgcacattgcgatttagatggtgacggtaaacttgatgtggacgaaatgacaagacaacatttgggattttggtattcggtcgacccaacttgtgaaggactctacggtggtgctgtaccttattaa

SEQ ID NO: 36 - protein, 198 aa, artificial sequence
  sequence derived by mutation of mitrocomin
MSMGSRYAVKLTTDFDNPKWIARHRHMFNFLDINSNGQINLNEMVHKASNIICKKLGATEEQTKRHQKCVEDFFGGAGLEYDKDTTWPEYIEGWKRLAKTELERHSKNQVTLIRLWGDALFDIIDKDRNGSVSLDEWIQYTHCAGIQQSRGQCEATFAHCDLDGDGKLDVDEMTRQHLGFWYSVDPTCEGLYGGAVPY

SEQ ID NO: 37 - DNA, 597 bp, artificial sequence
  sequence derived by mutation of mitrocomin
atgtcaatgggcagcagatacgcagtcaagcttacgactgactttgataatccaaaatggattgctcgacacaagcacatgttcaacttccttgacatcaattcaaatggccaaatcaatctgaatgaaatggtccataaggcttcaaacattatctgcaagaagcttggagcaacagaagaacaaaccaaacgtcatcaaaagtgtgtcgaagacttctttgggggagctggtttggaatatgacaaagataccacatggcctgagtacatcgaaggatggaagaggttggctaagactgaattggaaaggcattcaaagaatcaagtcacattgatccgattatggggtgatgctttgttcgacatcattgacaaagatagaaatggatcggtttcgttagacgaatggatccagtacactcattgtgctggcatccaacagtcacgtgggcaatgcgaagctacatttgcacattgcgatttagatggtgacggtaaacttgatgtggacgaaatgacaagacgacatttgggattttggtattcggtcgacccaacttgtgaaggactctacggtggtgctgtaccttattaa

SEQ ID NO: 38 - protein, 198 aa, artificial sequence
  sequence derived by mutation of mitrocomin
MSMGSRYAVKLTTDFDNPKWIARHKHMFNFLDINSNGQINLNEMVHKASNIICKKLGATEEQTKRHQKCVEDFFGGAGLEYDKDTTWPEYIEGWKRLAKTELERHSKNQVTLIRLWGDALFDIIDKDRNGSVSLDEWIQYTHCAGIQQSRGQCEATFAHCDLDGDGKLDVDEMTRRHLGFWYSVDPTCEGLYGGAVPY

SEQ ID NO: 39 - DNA, 597 bp, artificial sequence
  sequence derived by mutation of mitrocomin
atgtcaatgggcagcagatacgcagtcaagcttacgactgactttgataatccaaaatggattgctcgacacaagcacatgttcaacttccttgacatcaattcaaatggccaaatcaatctgaatgaaatggtccataaggcttcaaacattatctgcaagaagcttggagcaacagaagaacaaaccaaacgtcatcaaaagtgtgtcgaagacttctttgggggagctggtttggaatatgacaaagataccacatggcctgagtacatcgaaggatggaagaggttggctaagactgaattggaaaggcattcaaagaatcaagtcacattgatccgattatggggtgatgctttgttcgacatcattgacaaagatagaaatggatcggtttcgttagacgaatggatccagtacactcattgtgctggcatccaacagtcacgtgggcaatgcgaagctacatttgcacattgcgatttagatggtgacggtaaacttgatgtggacgaaatgacaagacaacatataggattttggtattcggtcgacccaacttgtgaaggactctacggtggtgctgtaccttattaa

SEQ ID NO: 40 - protein, 198 aa, artificial sequence
  sequence derived by mutation of mitrocomin
MSMGSRYAVKLTTDFDNPKWIARHKHMFNFLDINSNGQINLNEMVHKASNIICKKLGATEEQTKRHQKCVEDFFGGAGLEYDKDTTWPEYIEGWKRLAKTELERHSKNQVTLIRLWGDALFDIIDKDRNGSVSLDEWIQYTHCAGIQQSRGQCEATFAHCDLDGDGKLDVDEMTRQHIGFWYSVDPTCEGLYGGAVPY

SEQ ID NO: 41 - DNA, 597 bp, artificial sequence
  sequence derived by mutation of mitrocomin
atgtcaatgggcagcagatacgcagtcaagcttacgactgactttgataatccaaaatggattgctcgacacaggcacatgttcaacttccttgacatcaattcaaatggccaaatcaatctgaatgaaatggtccataaggcttcaaacattatctgcaagaagcttggagcaacagaagaacaaaccaaacgtcatcaaaagtgtgtcgaagacttctttgggggagctggtttggaatatgacaaagataccacatggcctgagtacatcgaaggatggaagaggttggctaagactgaattggaaaggcattcaaagaatcaagtcacattgatccgattatggggtgatgctttgttcgacatcattgacaaagatagaaatggatcggtttcgttagacgaatggatccagtacactcattgtgctggcatccaacagtcacgtgggcaatgcgaagctacatttgcacattgcgatttagatggtgacggtaaacttgatgtggacgaaatgacaagacgacatttgggattttggtattcggtcgacccaacttgtgaaggactctacggtggtgctgtaccttattaa

SEQ ID NO: 42 - protein, 198 aa, artificial sequence
  sequence derived by mutation of mitrocomin
MSMGSRYAVKLTTDFDNPKWIARHRHMFNFLDINSNGQINLNEMVHKASNIICKKLGATEEQTKRHQKCVEDFFGGAGLEYDKDTTWPEYIEGWKRLAKTELERHSKNQVTLIRLWGDALFDIIDKDRNGSVSLDEWIQYTHCAGIQQSRGQCEATFAHCDLDGDGKLDVDEMTRRHLGFWYSVDPTCEGLYGGAVPY

SEQ ID NO: 43 - DNA, 597 bp, artificial sequence
  sequence derived by mutation of mitrocomin
atgtcaatgggcagcagatacgcagtcaagcttacgactgactttgataatccaaaatggattgctcgacacaggcacatgttcaacttccttgacatcaattcaaatggccaaatcaatctgaatgaaatggtccataaggcttcaaacattatctgcaagaagcttggagcaacagaagaacaaaccaaacgtcatcaaaagtgtgtcgaagacttctttgggggagctggtttggaatatgacaaagataccacatggcctgagtacatcgaaggatggaagaggttggctaagactgaattggaaaggcattcaaagaatcaagtcacattgatccgattatggggtgatgctttgttcgacatcattgacaaagatagaaatggatcggtttcgttagacgaatggatccagtacactcattgtgctggcatccaacagtcacgtgggcaatgcgaagctacatttgcacattgcgatttagatggtgacggtaaacttgatgtggacgaaatgacaagacaacatataggattttggtattcggtcgacccaacttgtgaaggactctacggtggtgctgtaccttattaa

SEQ ID NO: 44 - protein, 198 aa, artificial sequence
  sequence derived by mutation of mitrocomin
MSMGSRYAVKLTTDFDNPKWIARHRHMFNFLDINSNGQINLNEMVHKASNIICKKLGATEEQTKRHQKCVEDFFGGAGLEYDKDTTWPEYIEGWKRLAKTELERHSKNQVTLIRLWGDALFDIIDKDRNGSVSLDEWIQYTHCAGIQQSRGQCEATFAHCDLDGDGKLDVDEMTRQHIGFWYSVDPTCEGLYGGAVPY

SEQ ID NO: 45 - DNA, 597 bp, artificial sequence
  sequence derived by mutation of mitrocomin
atgtcaatgggcagcagatacgcagtcaagcttacgactgactttgataatccaaaatggattgctcgacacaagcacatgttcaacttccttgacatcaattcaaatggccaaatcaatctgaatgaaatggtccataaggcttcaaacattatctgcaagaagcttggagcaacagaagaacaaaccaaacgtcatcaaaagtgtgtcgaagacttctttgggggagctggtttggaatatgacaaagataccacatggcctgagtacatcgaaggatggaagaggttggctaagactgaattggaaaggcattcaaagaatcaagtcacattgatccgattatggggtgatgctttgttcgacatcattgacaaagatagaaatggatcggtttcgttagacgaatggatccagtacactcattgtgctggcatccaacagtcacgtgggcaatgcgaagctacatttgcacattgcgatttagatggtgacggtaaacttgatgtggacgaaatgacaagacgacatataggattttggtattcggtcgacccaacttgtgaaggactctacggtggtgctgtaccttattaa

SEQ ID NO: 46 - protein, 198 aa, artificial sequence
  sequence derived by mutation of mitrocomin
MSMGSRYAVKLTTDFDNPKWIARHKHMFNFLDINSNGQINLNEMVHKASNIICKKLGATEEQTKRHQKCVEDFFGGAGLEYDKDTTWPEYIEGWKRLAKTELERHSKNQVTLIRLWGDALFDIIDKDRNGSVSLDEWIQYTHCAGIQQSRGQCEATFAHCDLDGDGKLDVDEMTRRHIGFWYSVDPTCEGLYGGAVPY

SEQ ID NO: 47 - DNA, 597 bp, artificial sequence
  sequence derived by mutation of mitrocomin
atgtcaatgggcagcagatacgcagtcaagcttacgactgactttgataatccaaaatggattgctcgacacaggcacatgttcaacttccttgacatcaattcaaatggccaaatcaatctgaatgaaatggtccataaggcttcaaacattatctgcaagaagcttggagcaacagaagaacaaaccaaacgtcatcaaaagtgtgtcgaagacttctttgggggagctggtttggaatatgacaaagataccacatggcctgagtacatcgaaggatggaagaggttggctaagactgaattggaaaggcattcaaagaatcaagtcacattgatccgattatggggtgatgctttgttcgacatcattgacaaagatagaaatggatcggtttcgttagacgaatggatccagtacactcattgtgctggcatccaacagtcacgtgggcaatgcgaagctacatttgcacattgcgatttagatggtgacggtaaacttgatgtggacgaaatgacaagacgacatataggattttggtattcggtcgacccaacttgtgaaggactctacggtggtgctgtaccttattaa

SEQ ID NO: 48 - protein, 198 aa, artificial sequence
  sequence derived by mutation of mitrocomin
MSMGSRYAVKLTTDFDNPKWIARHRHMFNFLDINSNGQINLNEMVHKASNIICKKLGATEEQTKRHQKCVEDFFGGAGLEYDKDTTWPEYIEGWKRLAKTELERHSKNQVTLIRLWGDALFDIIDKDRNGSVSLDEWIQYTHCAGIQQSRGQCEATFAHCDLDGDGKLDVDEMTRRHIGFWYSVDPTCEGLYGGAVPY

SEQ ID NO: 49 - DNA, 588 bp, Obelia longissima
  CDS (1)..(588)
atgtcttcaaaatacgcagttaaactcaagactgactttgataatccacgatggatcaaaagacacaagcacatgtttgatttcctcgacatcaatggaaatggaaaaatcaccctcgatgaaattgtgtccaaggcatctgatgacatatgtgccaagctcgaagccacaccagaacaaacaaaacgccatcaagtttgtgttgaagctttctttagaggatgtggaatggaatatggtaaagaaattgccttcccacaattcctcgatggatggaaacaattggcgacttcagaactcaagaaatgggcaagaaacgaacctactctcattcgtgaatggggagatgctgtctttgatattttcgacaaagatggaagtggtacaatcactttggacgaatggaaagcttatggaaaaatctctggtatctctccatcacaagaagattgtgaagcgacatttcgacattgcgatttggacaacagtggtgaccttgatgttgacgagatgacaagacaacatcttggattctggtacactttggacccagaagctgatggtctctatggcaacggagttccctaa

SEQ ID NO: 50 - protein, 195 aa, Obelia longissima
MSSKYAVKLKTDFDNPRWIKRHKHMFDFLDINGNGKITLDEIVSKASDDICAKLEATPEQTKRHQVCVEAFFRGCGMEYGKEIAFPQFLDGWKQLATSELKKWARNEPTLIREWGDAVFDIFDKDGSGTITLDEWKAYGKISGISPSQEDCEATFRHCDLDNSGDLDVDEMTRQHLGFWYTLDPEADGLYGNGVP

SEQ ID NO: 51 - DNA, 588 bp, artificial sequence
  sequence derived by mutation of obelin
atgtcttcaaaatacgcagttaaactcaagactgactttgataatccacgatggatcaaaagacacaggcacatgtttgatttcctcgacatcaatggaaatggaaaaatcaccctcgatgaaattgtgtccaaggcatctgatgacatatgtgccaagctcgaagccacaccagaacaaacaaaacgccatcaagtttgtgttgaagctttctttagaggatgtggaatggaatatggtaaagaaattgccttcccacaattcctcgatggatggaaacaattggcgacttcagaactcaagaaatgggcaagaaacgaacctactctcattcgtgaatggggagatgctgtctttgatattttcgacaaagatggaagtggtacaatcactttggacgaatggaaagcttatggaaaaatctctggtatctctccatcacaagaagattgtgaagcgacatttcgacattgcgatttggacaacagtggtgaccttgatgttgacgagatgacaagacaacatcttggattctggtacactttggacccagaagctgatggtctctatggcaacggagttccctaa

SEQ ID NO: 52 - protein, 195 aa, artificial sequence
  sequence derived by mutation of obelin
MSSKYAVKLKTDFDNPRWIKRHRHMFDFLDINGNGKITLDEIVSKASDDICAKLEATPEQTKRHQVCVEAFFRGCGMEYGKEIAFPQFLDGWKQLATSELKKWARNEPTLIREWGDAVFDIFDKDGSGTITLDEWKAYGKISGISPSQEDCEATFRHCDLDNSGDLDVDEMTRQHLGFWYTLDPEADGLYGNGVP

SEQ ID NO: 53 - DNA, 588 bp, artificial sequence
  sequence derived by mutation of obelin
atgtcttcaaaatacgcagttaaactcaagactgactttgataatccacgatggatcaaaagacacaagcacatgtttgatttcctcgacatcaatggaaatggaaaaatcaccctcgatgaaattgtgtccaaggcatctgatgacatatgtgccaagctcgaagccacaccagaacaaacaaaacgccatcaagtttgtgttgaagctttctttagaggatgtggaatggaatatggtaaagaaattgccttcccacaattcctcgatggatggaaacaattggcgacttcagaactcaagaaatgggcaagaaacgaacctactctcattcgtgaatggggagatgctgtctttgatattttcgacaaagatggaagtggtacaatcactttggacgaatggaaagcttatggaaaaatctctggtatctctccatcacaagaagattgtgaagcgacatttcgacattgcgatttggacaacagtggtgaccttgatgttgacgagatgacaagacgacatcttggattctggtacactttggacccagaagctgatggtctctatggcaacggagttccctaa

SEQ ID NO: 54 - protein, 195 aa, artificial sequence
  sequence derived by mutation of obelin
MSSKYAVKLKTDFDNPRWIKRHKHMFDFLDINGNGKITLDEIVSKASDDICAKLEATPEQTKRHQVCVEAFFRGCGMEYGKEIAFPQFLDGWKQLATSELKKWARNEPTLIREWGDAVFDIFDKDGSGTITLDEWKAYGKISGISPSQEDCEATFRHCDLDNSGDLDVDEMTRRHLGFWYTLDPEADGLYGNGVP

SEQ ID NO: 55 - DNA, 588 bp, artificial sequence
  sequence derived by mutation of obelin
atgtcttcaaaatacgcagttaaactcaagactgactttgataatccacgatggatcaaaagacacaagcacatgtttgatttcctcgacatcaatggaaatggaaaaatcaccctcgatgaaattgtgtccaaggcatctgatgacatatgtgccaagctcgaagccacaccagaacaaacaaaacgccatcaagtttgtgttgaagctttctttagaggatgtggaatggaatatggtaaagaaattgccttcccacaattcctcgatggatggaaacaattggcgacttcagaactcaagaaatgggcaagaaacgaacctactctcattcgtgaatggggagatgctgtctttgatattttcgacaaagatggaagtggtacaatcactttggacgaatggaaagcttatggaaaaatctctggtatctctccatcacaagaagattgtgaagcgacatttcgacattgcgatttggacaacagtggtgaccttgatgttgacgagatgacaagacaacatattggattctggtacactttggacccagaagctgatggtctctatggcaacggagttccctaa

SEQ ID NO: 56 - protein, 195 aa, artificial sequence
  sequence derived by mutation of obelin
MSSKYAVKLKTDFDNPRWIKRHKHMFDFLDINGNGKITLDEIVSKASDDICAKLEATPEQTKRHQVCVEAFFRGCGMEYGKEIAFPQFLDGWKQLATSELKKWARNEPTLIREWGDAVFDIFDKDGSGTITLDEWKAYGKISGISPSQEDCEATFRHCDLDNSGDLDVDEMTRQHIGFWYTLDPEADGLYGNGVP

SEQ ID NO: 57 - DNA, 588 bp, artificial sequence
  sequence derived by mutation of obelin
atgtcttcaaaatacgcagttaaactcaagactgactttgataatccacgatggatcaaaagacacaggcacatgtttgatttcctcgacatcaatggaaatggaaaaatcaccctcgatgaaattgtgtccaaggcatctgatgacatatgtgccaagctcgaagccacaccagaacaaacaaaacgccatcaagtttgtgttgaagctttctttagaggatgtggaatggaatatggtaaagaaattgccttcccacaattcctcgatggatggaaacaattggcgacttcagaactcaagaaatgggcaagaaacgaacctactctcattcgtgaatggggagatgctgtctttgatattttcgacaaagatggaagtggtacaatcactttggacgaatggaaagcttatggaaaaatctctggtatctctccatcacaagaagattgtgaagcgacatttcgacattgcgatttggacaacagtggtgaccttgatgttgacgagatgacaagacgacatcttggattctggtacactttggacccagaagctgatggtctctatggcaacggagttccctaa

SEQ ID NO: 58 - protein, 195 aa, artificial sequence
  sequence derived by mutation of obelin
MSSKYAVKLKTDFDNPRWIKRHRHMFDFLDINGNGKITLDEIVSKASDDICAKLEATPEQTKRHQVCVEAFFRGCGMEYGKEIAFPQFLDGWKQLATSELKKWARNEPTLIREWGDAVFDIFDKDGSGTITLDEWKAYGKISGISPSQEDCEATFRHCDLDNSGDLDVDEMTRRHLGFWYTLDPEADGLYGNGVP

SEQ ID NO: 59 - DNA, 588 bp, artificial sequence
  sequence derived by mutation of obelin
atgtcttcaaaatacgcagttaaactcaagactgactttgataatccacgatggatcaaaagacacaggcacatgtttgatttcctcgacatcaatggaaatggaaaaatcaccctcgatgaaattgtgtccaaggcatctgatgacatatgtgccaagctcgaagccacaccagaacaaacaaaacgccatcaagtttgtgttgaagctttctttagaggatgtggaatggaatatggtaaagaaattgccttcccacaattcctcgatggatggaaacaattggcgacttcagaactcaagaaatgggcaagaaacgaacctactctcattcgtgaatggggagatgctgtctttgatattttcgacaaagatggaagtggtacaatcactttggacgaatggaaagcttatggaaaaatctctggtatctctccatcacaagaagattgtgaagcgacatttcgacattgcgatttggacaacagtggtgaccttgatgttgacgagatgacaagacaacatattggattctggtacactttggacccagaagctgatggtctctatggcaacggagttccctaa

SEQ ID NO: 60 - protein, 195 aa, artificial sequence
  sequence derived by mutation of obelin
MSSKYAVKLKTDFDNPRWIKRHRHMFDFLDINGNGKITLDEIVSKASDDICAKLEATPEQTKRHQVCVEAFFRGCGMEYGKEIAFPQFLDGWKQLATSELKKWARNEPTLIREWGDAVFDIFDKDGSGTITLDEWKAYGKISGISPSQEDCEATFRHCDLDNSGDLDVDEMTRQHIGFWYTLDPEADGLYGNGVP

SEQ ID NO: 61 - DNA, 588 bp, artificial sequence
  sequence derived by mutation of obelin
atgtcttcaaaatacgcagttaaactcaagactgactttgataatccacgatggatcaaaagacacaagcacatgtttgatttcctcgacatcaatggaaatggaaaaatcaccctcgatgaaattgtgtccaaggcatctgatgacatatgtgccaagctcgaagccacaccagaacaaacaaaacgccatcaagtttgtgttgaagctttctttagaggatgtggaatggaatatggtaaagaaattgccttcccacaattcctcgatggatggaaacaattggcgacttcagaactcaagaaatgggcaagaaacgaacctactctcattcgtgaatggggagatgctgtctttgatattttcgacaaagatggaagtggtacaatcactttggacgaatggaaagcttatggaaaaatctctggtatctctccatcacaagaagattgtgaagcgacatttcgacattgcgatttggacaacagtggtgaccttgatgttgacgagatgacaagacgacatattggattctggtacactttggacccagaagctgatggtctctatggcaacggagttccctaa

SEQ ID NO: 62 - protein, 195 aa, artificial sequence
  sequence derived by mutation of obelin
MSSKYAVKLKTDFDNPRWIKRHKHMFDFLDINGNGKITLDEIVSKASDDICAKLEATPEQTKRHQVCVEAFFRGCGMEYGKEIAFPQFLDGWKQLATSELKKWARNEPTLIREWGDAVFDIFDKDGSGTITLDEWKAYGKISGISPSQEDCEATFRHCDLDNSGDLDVDEMTRRHIGFWYTLDPEADGLYGNGVP

SEQ ID NO: 63 - DNA, 588 bp, artificial sequence
  sequence derived by mutation of obelin
atgtcttcaaaatacgcagttaaactcaagactgactttgataatccacgatggatcaaaagacacaggcacatgtttgatttcctcgacatcaatggaaatggaaaaatcaccctcgatgaaattgtgtccaaggcatctgatgacatatgtgccaagctcgaagccacaccagaacaaacaaaacgccatcaagtttgtgttgaagctttctttagaggatgtggaatggaatatggtaaagaaattgccttcccacaattcctcgatggatggaaacaattggcgacttcagaactcaagaaatgggcaagaaacgaacctactctcattcgtgaatggggagatgctgtctttgatattttcgacaaagatggaagtggtacaatcactttggacgaatggaaagcttatggaaaaatctctggtatctctccatcacaagaagattgtgaagcgacatttcgacattgcgatttggacaacagtggtgaccttgatgttgacgagatgacaagacgacatattggattctggtacactttggacccagaagctgatggtctctatggcaacggagttccctaa

SEQ ID NO: 64 - protein, 195 aa, artificial sequence
  sequence derived by mutation of obelin
MSSKYAVKLKTDFDNPRWIKRHRHMFDFLDINGNGKITLDEIVSKASDDICAKLEATPEQTKRHQVCVEAFFRGCGMEYGKEIAFPQFLDGWKQLATSELKKWARNEPTLIREWGDAVFDIFDKDGSGTITLDEWKAYGKISGISPSQEDCEATFRHCDLDNSGDLDVDEMTRRHIGFWYTLDPEADGLYGNGVP

SEQ ID NO: 65 - DNA, 600 bp, artificial sequence
  sequence derived by mutation of aequorin
atgctttatgatgttcctgattatgctagcctcaaacttacatcagacttcgacaacccaagatggattggacgacacaggcatatgttcaatttccttgatgtcaaccacaatggaaaaatctctcttgacgggatggtctacaaggcatctgatattgtcatcaataaccttggagcaacacctgagcaagccaaacgacacaaagatgctgtagaagccttcttcggaggagctggaatgaaatatggtgtggaaactgattggcctgcatatattgaaggatggaaaaaattggctactgatgaattggagaaatacgccaaaaacgaaccaacgctcatccgtatatggggtgatgctttgtttgatatcgttgacaaagatcaaaatggagccattacactggatgaatggaaagcatacaccaaagctgctggtatcatccaatcatcagaagattgcgaggaaacattcagagtgtgcgatattgatgaaagtggacaactcgatgttgatgagatgacaagacaacatttaggattttggtacaccatggatcctgcttgcgaaaagctctacggtggagctgtcccctaa

SEQ ID NO: 66 - protein, 199 aa, artificial sequence
  sequence derived by mutation of aequorin
MLYDVPDYASLKLTSDFDNPRWIGRHRHMFNFLDVNHNGKISLDGMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGQLDVDEMTRQHLGFWYTMDPACEKLYGGAVP

SEQ ID NO: 67 - DNA, 600 bp, artificial sequence
  sequence derived by mutation of aequorin
atgctttatgatgttcctgattatgctagcctcaaacttacatcagacttcgacaacccaagatggattggacgacacaagcatatgttcaatttccttgatgtcaaccacaatggaaaaatctctcttgacgagatggtctacaaggcatctgatattgccatcaataaccttggagcaacacctgagcaagccaaacgacacaaagatgctgtagaagccttcttcggaggagctggaatgaaatatggtgtggaaactgattggcctgcatatattgaaggatggaaaaaattggctactgatgaattggagaaatacgccaaaaacgaaccaacgctcatccgtatatggggtgatgctttgtttgatatcgttgacaaagatcaaaatggagccattacactggatgaatggaaagcatacaccaaagctgctggtatcatccaatcatcagaagattgcgaggaaacattcagagtgtgcgatattgatgaaagtggacaactcgatgttgatgagatgacaagacgacatttaggattttggtacaccatggatcctgcttgcgaaaagctctacggtggagctgtcccctaa

SEQ ID NO: 68 - protein, 199 aa, artificial sequence
  sequence derived by mutation of aequorin
MLYDVPDYASLKLTSDFDNPRWIGRHKHMFNFLDVNHNGKISLDEMVYKASDIAINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGQLDVDEMTRRHLGFWYTMDPACEKLYGGAVP

SEQ ID NO: 69 - DNA, 600 bp, artificial sequence
  sequence derived by mutation of aequorin
atgctttatgatgttcctgattatgctagcctcaaacttacatcagacttcgacaacccaagatggattggacgacacaagcatatgttcaatttccttgatgtcaaccacaatggaaaaatctctcttgacgagatggtctacaaggcatctgatattgtcatcaataaccttggagcaacacctgagcaagccaaacgacacaaagatgctgtagaagccttcttcggaggagctggaatgaaatatggtgtggaaactgattggcctgcatatattgaaggatggaaaaaattggctactgatgaattggagaaatacgccaaaaacgaaccaacgctcatccgtatatggggtgatgctttgtttgatatcgttggcaaagatcaaaatggagccattacactggatgaatggaaagcatacaccaaagctgctggtatcatccaatcatcagaagattgcgaggaaacattcagagtgtgcgatattgatgaaagtggacaactcgatgttgatgagatgacaagacaacatataggattttggtacaccatggatcctgcttgcgaaaagctctacggtggagctgtcccctaa

SEQ ID NO: 70 - protein, 199 aa, artificial sequence
  sequence derived by mutation of aequorin
MLYDVPDYASLKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVGKDQNGAITLDEWKAYTKAAGIIQSSEDCEETFRVCDIDESGQLDVDEMTRQHIGFWYTMDPACEKLYGGAVP

SEQ ID NO: 71 - DNA, 600 bp, artificial sequence
  sequence derived by mutation of aequorin
atgctttatgatgttcctgattatgctagcctcaaacttacatcagacttcgacaacccaagatggattggacgacacaagcatatgttcaatttccttgatgtcaaccacaatggaaaaatctctcttgacgagatggtctacaaggcatctgatattgtcatcaataaccttggagcaacacctgagcaagccaaacgacacaaagatgctgtagaagccttcttcggaggagctggaatgaaatatggtgtggaaactgattggcctgcatatattgaaggatggaaaaaattggctactgatgaattggagaaatacgccaaaaacgaaccaacgctcatccgtatatggggtgatgctttgtttgatatcgttgacaaagatcaaaatggagccattacactggatggatggaaagcatacaccaaagctgctggtatcatccaatcatcagaagattgcgaggaaacattcagagtgtgcgatattgatgaaagtggacaactcgatgttgatgagatgacaagacaacatataggattttggtacaccatggatcctgcttgcgaaaagctctacggtggagctgtcccctaa

SEQ ID NO: 72 - protein, 199 aa, artificial sequence
  sequence derived by mutation of aequorin
MLYDVPDYASLKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDGWKAYTKAAGIIQSSEDCEETFRVCDIDESGQLDVDEMTRQHIGFWYTMDPACEKLYGGAVP

SEQ ID NO: 73 - DNA, 600 bp, artificial sequence
  sequence derived by mutation of aequorin
atgctttatgatgttcctgattatgctagcctcaaacttacatcagacttcgacaacccaagatggattggacgacacaagcatatgttcaatttccttgatgtcaaccacaatggaaaaatctctcttgacgagatggtctacaaggcatctgatattgtcatcaataaccttggagcaacacctgagcaagccaaacgacacaaagatgctgtagaagccttcttcggaggagctggaatgaaatatggtgtggaaactgattggcctgcatatattgaaggatggaaaaaattggctactgatgaattggagaaatacgccaaaaacgaaccaacgctcatccgtatatggggtgatgctttgtttgatatcgttgacaaagatcaaaatggagccattacactggatgaatggaaagcatacaccaaagctgctggtatcatccaatcatcagaagattgcgaggaaacatccagagtgtgcgatattgatgaaagtggacaactcgatgttgatgagatgacaagacaacatataggattttggtacaccatggatcctgcttgcgaaaagctctacggtggagctgtcccctaa

SEQ ID NO: 74 - protein, 199 aa, artificial sequence
  sequence derived by mutation of aequorin
MLYDVPDYASLKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETSRVCDIDESGQLDVDEMTRQHIGFWYTMDPACEKLYGGAVP

SEQ ID NO: 75 - DNA, 600 bp, artificial sequence
  sequence derived by mutation of aequorin
atgctttatgatgttcctgattatgctagcctcaaacttacatcagacttcgacaacccaagatggattggacgacacaagcatatgttcaatttccttgatgtcaaccacaatggaaaaatctctcttgacgagatggtctacaaggcatctgatattgtcatcaataaccttggagcaacacctgagcaagccaaacgacacaaagatgctgtagaagccttcttcggaggagctggaatgaaatatggtgtggaaactgattggcctgcatatattgaaggatggaaaaaattggctactgatgaattggagaaatacgccaaaaacgaaccaacgctcatccgtatatggggtgatgctttgtttgatatcgttgacaaagatcaaaatggagccattacactggatgaatggaaagcatacaccaaagctgctggtatcatccaatcatcagaagattgcgaggaaacattcagagtgtgcggtattgatgaaagtggacaactcgatgttgatgagatgacaagacaacatataggattttggtacaccatggatcctgcttgcgaaaagctctacggtggagctgtcccctaa

SEQ ID NO: 76 - protein, 199 aa, artificial sequence
  sequence derived by mutation of aequorin
MLYDVPDYASLKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCGIDESGQLDVDEMTRQHIGFWYTMDPACEKLYGGAVP

SEQ ID NO: 77 - DNA, 597 bp, artificial sequence
  sequence derived by mutation of clytin
atggctgacactgcatcaaaatacgccgtcaaactcagacccaacttcgacaacccaaaatgggtcaacagacacaaatttatgttcaactttttggacattaacggcgacggaaaaatcactttggatggaatcgtctccaaagcttcggatgacatttgcgccaaacttggagcaacaccagaacagaccaaacgtcaccaggatgctgtcgaagctttcttcaaaaagattggtatggattatggtaaagaagtcgaattcccagcttttgttgatggatggaaagaactggccaattatgacttgaaactttggtctcaaaacaagaaatctttgatccgcgactggggagaagctgttttcgacatttttgacaaagacggaagtggctcaatcagtttggacgaatggaaggcttatggacgaatctctggaatctgctcatcagacgaagacgccgaaaagaccttcaaacattgcgatttggacaacagtggcaaacttgatgttgatgagatgaccagacaacatttgggattctggtacaccttggaccccaacgctgatggtctttacggcaattttgttccttaa

SEQ ID NO: 78 - protein, 198 aa, artificial sequence
  sequence derived by mutation of clytin
MADTASKYAVKLRPNFDNPKWVNRHKFMFNFLDINGDGKITLDGIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGKLDVDEMTRQHLGFWYTLDPNADGLYGNFVP

SEQ ID NO: 79 - DNA, 597 bp, artificial sequence
  sequence derived by mutation of clytin
atggctgacactgcatcaaaatacgccgtcaaactcagacccaacttcgacaacccaaaatgggtcaacagacacaaatttatgttcaactttttggacattaacggcgacggaaaaatcactttggatgaaatcgtctccaaagcttcggatgacatttgcgccaaacttggagcaacaccagaacagaccaaacgtcaccaggatgctgtcgaagctttcttcaaaaagattggtatggattatggtaaagaagtcgaattcccagcttttgttgatggatggaaagaactggccaattatgacttgaaactttggtctcaaaacaagaaatctttgatccgcgactggggagaagctgttttcgacatttttggcaaagacggaagtggctcaatcagtttggacgaatggaaggcttatggacgaatctctggaatctgctcatcagacgaagacgccgaaaagaccttcaaacattgcgatttggacaacagtggcaaacttgatgttgatgagatgaccagacaacatttgggattctggtacaccttggaccccaacgctgatggtctttacggcaattttgttccttaa

SEQ ID NO: 80 - protein, 198 aa, artificial sequence
  sequence derived by mutation of clytin
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFGKDGSGSISLDEWKAYGRISGICSSDEDAEKTFKHCDLDNSGKLDVDEMTRQHLGFWYTLDPNADGLYGNFVP

SEQ ID NO: 81 - DNA, 597 bp, artificial sequence
  sequence derived by mutation of clytin
atggctgacactgcatcaaaatacgccgtcaaactcagacccaacttcgacaacccaaaatgggtcaacagacacaaatttatgttcaactttttggacattaacggcgacggaaaaatcactttggatgaaatcgtctccaaagcttcggatgacatttgcgccaaacttggagcaacaccagaacagaccaaacgtcaccaggatgctgtcgaagctttcttcaaaaagattggtatggattatggtaaagaagtcgaattcccagcttttgttgatggatggaaagaactggccaattatgacttgaaactttggtctcaaaacaagaaatctttgatccgcgactggggagaagctgttttcgacatttttgacaaagacggaagtggctcaatcagtttggacggatggaaggcttatggacgaatctctggaatctgctcatcagacgaagacgccgaaaagaccttcaaacattgcgatttggacaacagtggcaaacttgatgttgatgagatgaccagacaacatttgggattctggtacaccttggaccccaacgctgatggtctttacggcaattttgttccttaa

SEQ ID NO: 82 - protein, 198 aa, artificial sequence
  sequence derived by mutation of clytin
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDGWKAYGRISGICSSDEDAEKTFKHCDLDNSGKLDVDEMTRQHLGFWYTLDPNADGLYGNFVP

SEQ ID NO: 83 - DNA, 597 bp, artificial sequence
  sequence derived by mutation of clytin
atggctgacactgcatcaaaatacgccgtcaaactcagacccaacttcgacaacccaaaatgggtcaacagacacaaatttatgttcaactttttggacattaacggcgacggaaaaatcactttggatgaaatcgtctccaaagcttcggatgacatttgcgccaaacttggagcaacaccagaacagaccaaacgtcaccaggatgctgtcgaagctttcttcaaaaagattggtatggattatggtaaagaagtcgaattcccagcttttgttgatggatggaaagaactggccaattatgacttgaaactttggtctcaaaacaagaaatctttgatccgcgactggggagaagctgttttcgacatttttgacaaagacggaagtggctcaatcagtttggacgaatggaaggcttatggacgaatctctggaatctgctcatcagacgaagacgccgaaaagacctccaaacattgcgatttggacaacagtggcaaacttgatgttgatgagatgaccagacaacatttgggattctggtacaccttggaccccaacgctgatggtctttacggcaattttgttccttaa

SEQ ID NO: 84 - protein, 198 aa, artificial sequence
  sequence derived by mutation of clytin
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTSKHCDLDNSGKLDVDEMTRQHLGFWYTLDPNADGLYGNFVP

SEQ ID NO: 85 - DNA, 597 bp, artificial sequence
  sequence derived by mutation of clytin
atggctgacactgcatcaaaatacgccgtcaaactcagacccaacttcgacaacccaaaatgggtcaacagacacaaatttatgttcaactttttggacattaacggcgacggaaaaatcactttggatgaaatcgtctccaaagcttcggatgacatttgcgccaaacttggagcaacaccagaacagaccaaacgtcaccaggatgctgtcgaagctttcttcaaaaagattggtatggattatggtaaagaagtcgaattcccagcttttgttgatggatggaaagaactggccaattatgacttgaaactttggtctcaaaacaagaaatctttgatccgcgactggggagaagctgttttcgacatttttgacaaagacggaagtggctcaatcagtttggacgaatggaaggcttatggacgaatctctggaatctgctcatcagacgaagacgccgaaaagaccttcaaacattgcggtttggacaacagtggcaaacttgatgttgatgagatgaccagacaacatttgggattctggtacaccttggaccccaacgctgatggtctttacggcaattttgttccttaa

SEQ ID NO: 86 - protein, 198 aa, artificial sequence
  sequence derived by mutation of clytin
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCGLDNSGKLDVDEMTRQHLGFWYTLDPNADGLYGNFVP

SEQ ID NO: 87 - DNA, 597 bp, artificial sequence
  sequence derived by mutation of mitrocomin
atgtcaatgggcagcagatacgcagtcaagcttacgactgactttgataatccaaaatggattgctcgacacaagcacatgttcaacttccttgacatcaattcaaatggccaaatcaatctgaatggaatggtccataaggcttcaaacattatctgcaagaagcttggagcaacagaagaacaaaccaaacgtcatcaaaagtgtgtcgaagacttctttgggggagctggtttggaatatgacaaagataccacatggcctgagtacatcgaaggatggaagaggttggctaagactgaattggaaaggcattcaaagaatcaagtcacattgatccgattatggggtgatgctttgttcgacatcattgacaaagatagaaatggatcggtttcgttagacgaatggatccagtacactcattgtgctggcatccaacagtcacgtgggcaatgcgaagctacatttgcacattgcgatttagatggtgacggtaaacttgatgtggacgaaatgacaagacaacatttgggattttggtattcggtcgacccaacttgtgaaggactctacggtggtgctgtaccttattaa

SEQ ID NO: 88 - protein, 198 aa, artificial sequence
  sequence derived by mutation of mitrocomin
MSMGSRYAVKLTTDFDNPKWIARHKHMFNFLDINSNGQINLNGMVHKASNIICKKLGATEEQTKRHQKCVEDFFGGAGLEYDKDTTWPEYIEGWKRLAKTELERHSKNQVTLIRLWGDALFDIIDKDRNGSVSLDEWIQYTHCAGIQQSRGQCEATFAHCDLDGDGKLDVDEMTRQHLGFWYSVDPTCEGLYGGAVPY

SEQ ID NO: 89 - DNA, 597 bp, artificial sequence
  sequence derived by mutation of mitrocomin
atgtcaatgggcagcagatacgcagtcaagcttacgactgactttgataatccaaaatggattgctcgacacaagcacatgttcaacttccttgacatcaattcaaatggccaaatcaatctgaatgaaatggtccataaggcttcaaacattatctgcaagaagcttggagcaacagaagaacaaaccaaacgtcatcaaaagtgtgtcgaagacttctttgggggagctggtttggaatatgacaaagataccacatggcctgagtacatcgaaggatggaagaggttggctaagactgaattggaaaggcattcaaagaatcaagtcacattgatccgattatggggtgatgctttgttcgacatcattggcaaagatagaaatggatcggtttcgttagacgaatggatccagtacactcattgtgctggcatccaacagtcacgtgggcaatgcgaagctacatttgcacattgcgatttagatggtgacggtaaacttgatgtggacgaaatgacaagacaacatttgggattttggtattcggtcgacccaacttgtgaaggactctacggtggtgctgtaccttattaa

SEQ ID NO: 90 - protein, 198 aa, artificial sequence
  sequence derived by mutation of mitrocomin
MSMGSRYAVKLTTDFDNPKWIARHKHMFNFLDINSNGQINLNEMVHKASNIICKKLGATEEQTKRHQKCVEDFFGGAGLEYDKDTTWPEYIEGWKRLAKTELERHSKNQVTLIRLWGDALFDIIGKDRNGSVSLDEWIQYTHCAGIQQSRGQCEATFAHCDLDGDGKLDVDEMTRQHLGFWYSVDPTCEGLYGGAVPY

SEQ ID NO: 91 - DNA, 597 bp, artificial sequence
  sequence derived by mutation of mitrocomin
atgtcaatgggcagcagatacgcagtcaagcttacgactgactttgataatccaaaatggattgctcgacacaagcacatgttcaacttccttgacatcaattcaaatggccaaatcaatctgaatgaaatggtccataaggcttcaaacattatctgcaagaagcttggagcaacagaagaacaaaccaaacgtcatcaaaagtgtgtcgaagacttctttgggggagctggtttggaatatgacaaagataccacatggcctgagtacatcgaaggatggaagaggttggctaagactgaattggaaaggcattcaaagaatcaagtcacattgatccgattatggggtgatgctttgttcgacatcattgacaaagatagaaatggatcggtttcgttagacggatggatccagtacactcattgtgctggcatccaacagtcacgtgggcaatgcgaagctacatttgcacattgcgatttagatggtgacggtaaacttgatgtggacgaaatgacaagacaacatttgggattttggtattcggtcgacccaacttgtgaaggactctacggtggtgctgtaccttattaa

SEQ ID NO: 92 - protein, 198 aa, artificial sequence
  sequence derived by mutation of mitrocomin
MSMGSRYAVKLTTDFDNPKWIARHKHMFNFLDINSNGQINLNEMVHKASNIICKKLGATEEQTKRHQKCVEDFFGGAGLEYDKDTTWPEYIEGWKRLAKTELERHSKNQVTLIRLWGDALFDIIDKDRNGSVSLDGWIQYTHCAGIQQSRGQCEATFAHCDLDGDGKLDVDEMTRQHLGFWYSVDPTCEGLYGGAVPY

SEQ ID NO: 93 - DNA, 597 bp, artificial sequence
  sequence derived by mutation of mitrocomin
atgtcaatgggcagcagatacgcagtcaagcttacgactgactttgataatccaaaatggattgctcgacacaagcacatgttcaacttccttgacatcaattcaaatggccaaatcaatctgaatgaaatggtccataaggcttcaaacattatctgcaagaagcttggagcaacagaagaacaaaccaaacgtcatcaaaagtgtgtcgaagacttctttgggggagctggtttggaatatgacaaagataccacatggcctgagtacatcgaaggatggaagaggttggctaagactgaattggaaaggcattcaaagaatcaagtcacattgatccgattatggggtgatgctttgttcgacatcattgacaaagatagaaatggatcggtttcgttagacgaatggatccagtacactcattgtgctggcatccaacagtcacgtgggcaatgcgaagctacatccgcacattgcgatttagatggtgacggtaaacttgatgtggacgaaatgacaagacaacatttgggattttggtattcggtcgacccaacttgtgaaggactctacggtggtgctgtaccttattaa

SEQ ID NO: 94 - protein, 198 aa, artificial sequence
  sequence derived by mutation of mitrocomin
MSMGSRYAVKLTTDFDNPKWIARHKHMFNFLDINSNGQINLNEMVHKASNIICKKLGATEEQTKRHQKCVEDFFGGAGLEYDKDTTWPEYIEGWKRLAKTELERHSKNQVTLIRLWGDALFDIIDKDRNGSVSLDEWIQYTHCAGIQQSRGQCEATSAHCDLDGDGKLDVDEMTRQHLGFWYSVDPTCEGLYGGAVPY

SEQ ID NO: 95 - DNA, 597 bp, artificial sequence
  sequence derived by mutation of mitrocomin
atgtcaatgggcagcagatacgcagtcaagcttacgactgactttgataatccaaaatggattgctcgacacaagcacatgttcaacttccttgacatcaattcaaatggccaaatcaatctgaatgaaatggtccataaggcttcaaacattatctgcaagaagcttggagcaacagaagaacaaaccaaacgtcatcaaaagtgtgtcgaagacttctttgggggagctggtttggaatatgacaaagataccacatggcctgagtacatcgaaggatggaagaggttggctaagactgaattggaaaggcattcaaagaatcaagtcacattgatccgattatggggtgatgctttgttcgacatcattgacaaagatagaaatggatcggtttcgttagacgaatggatccagtacactcattgtgctggcatccaacagtcacgtgggcaatgcgaagctacatttgcacattgcggtttagatggtgacggtaaacttgatgtggacgaaatgacaagacaacatttgggattttggtattcggtcgacccaacttgtgaaggactctacggtggtgctgtaccttattaa

SEQ ID NO: 96 - protein, 198 aa, artificial sequence
  sequence derived by mutation of mitrocomin
MSMGSRYAVKLTTDFDNPKWIARHKHMFNFLDINSNGQINLNEMVHKASNIICKKLGATEEQTKRHQKCVEDFFGGAGLEYDKDTTWPEYIEGWKRLAKTELERHSKNQVTLIRLWGDALFDIIDKDRNGSVSLDEWIQYTHCAGIQQSRGQCEATFAHCGLDGDGKLDVDEMTRQHLGFWYSVDPTCEGLYGGAVPY

SEQ ID NO: 97 - DNA, 588 bp, artificial sequence
  sequence derived by mutation of obelin
atgtcttcaaaatacgcagttaaactcaagactgactttgataatccacgatggatcaaaagacacaagcacatgtttgatttcctcgacatcaatggaaatggaaaaatcaccctcgatggaattgtgtccaaggcatctgatgacatatgtgccaagctcgaagccacaccagaacaaacaaaacgccatcaagtttgtgttgaagctttctttagaggatgtggaatggaatatggtaaagaaattgccttcccacaattcctcgatggatggaaacaattggcgacttcagaactcaagaaatgggcaagaaacgaacctactctcattcgtgaatggggagatgctgtctttgatattttcgacaaagatggaagtggtacaatcactttggacgaatggaaagcttatggaaaaatctctggtatctctccatcacaagaagattgtgaagcgacatttcgacattgcgatttggacaacagtggtgaccttgatgttgacgagatgacaagacaacatcttggattctggtacactttggacccagaagctgatggtctctatggcaacggagttccctaa

SEQ ID NO: 98 - protein, 195 aa, artificial sequence
  sequence derived by mutation of obelin
MSSKYAVKLKTDFDNPRWIKRHKHMFDFLDINGNGKITLDGIVSKASDDICAKLEATPEQTKRHQVCVEAFFRGCGMEYGKEIAFPQFLDGWKQLATSELKKWARNEPTLIREWGDAVFDIFDKDGSGTITLDEWKAYGKISGISPSQEDCEATFRHCDLDNSGDLDVDEMTRQHLGFWYTLDPEADGLYGNGVP

SEQ ID NO: 99 - DNA, 588 bp, artificial sequence
  sequence derived by mutation of obelin
atgtcttcaaaatacgcagttaaactcaagactgactttgataatccacgatggatcaaaagacacaagcacatgtttgatttcctcgacatcaatggaaatggaaaaatcaccctcgatgaaattgtgtccaaggcatctgatgacatatgtgccaagctcgaagccacaccagaacaaacaaaacgccatcaagtttgtgttgaagctttctttagaggatgtggaatggaatatggtaaagaaattgccttcccacaattcctcgatggatggaaacaattggcgacttcagaactcaagaaatgggcaagaaacgaacctactctcattcgtgaatggggagatgctgtctttgatattttcggcaaagatggaagtggtacaatcactttggacgaatggaaagcttatggaaaaatctctggtatctctccatcacaagaagattgtgaagcgacatttcgacattgcgatttggacaacagtggtgaccttgatgttgacgagatgacaagacaacatcttggattctggtacactttggacccagaagctgatggtctctatggcaacggagttccctaa

SEQ ID NO: 100 - protein, 195 aa, artificial sequence
  sequence derived by mutation of obelin
MSSKYAVKLKTDFDNPRWIKRHKHMFDFLDINGNGKITLDEIVSKASDDICAKLEATPEQTKRHQVCVEAFFRGCGMEYGKEIAFPQFLDGWKQLATSELKKWARNEPTLIREWGDAVFDIFGKDGSGTITLDEWKAYGKISGISPSQEDCEATFRHCDLDNSGDLDVDEMTRQHLGFWYTLDPEADGLYGNGVP

SEQ ID NO: 101 - DNA, 588 bp, artificial sequence
  sequence derived by mutation of obelin
atgtcttcaaaatacgcagttaaactcaagactgactttgataatccacgatggatcaaaagacacaagcacatgtttgatttcctcgacatcaatggaaatggaaaaatcaccctcgatgaaattgtgtccaaggcatctgatgacatatgtgccaagctcgaagccacaccagaacaaacaaaacgccatcaagtttgtgttgaagctttctttagaggatgtggaatggaatatggtaaagaaattgccttcccacaattcctcgatggatggaaacaattggcgacttcagaactcaagaaatgggcaagaaacgaacctactctcattcgtgaatggggagatgctgtctttgatattttcgacaaagatggaagtggtacaatcactttggacggatggaaagcttatggaaaaatctctggtatctctccatcacaagaagattgtgaagcgacatttcgacattgcgatttggacaacagtggtgaccttgatgttgacgagatgacaagacaacatcttggattctggtacactttggacccagaagctgatggtctctatggcaacggagttccctaa

SEQ ID NO: 102 - protein, 195 aa, artificial sequence
  sequence derived by mutation of obelin
MSSKYAVKLKTDFDNPRWIKRHKHMFDFLDINGNGKITLDEIVSKASDDICAKLEATPEQTKRHQVCVEAFFRGCGMEYGKEIAFPQFLDGWKQLATSELKKWARNEPTLIREWGDAVFDIFDKDGSGTITLDGWKAYGKISGISPSQEDCEATFRHCDLDNSGDLDVDEMTRQHLGFWYTLDPEADGLYGNGVP

SEQ ID NO: 103 - DNA, 588 bp, artificial sequence
  sequence derived by mutation of obelin
atgtcttcaaaatacgcagttaaactcaagactgactttgataatccacgatggatcaaaagacacaagcacatgtttgatttcctcgacatcaatggaaatggaaaaatcaccctcgatgaaattgtgtccaaggcatctgatgacatatgtgccaagctcgaagccacaccagaacaaacaaaacgccatcaagtttgtgttgaagctttctttagaggatgtggaatggaatatggtaaagaaattgccttcccacaattcctcgatggatggaaacaattggcgacttcagaactcaagaaatgggcaagaaacgaacctactctcattcgtgaatggggagatgctgtctttgatattttcgacaaagatggaagtggtacaatcactttggacgaatggaaagcttatggaaaaatctctggtatctctccatcacaagaagattgtgaagcgacatcccgacattgcgatttggacaacagtggtgaccttgatgttgacgagatgacaagacaacatcttggattctggtacactttggacccagaagctgatggtctctatggcaacggagttccctaa

SEQ ID NO: 104 - protein, 195 aa, artificial sequence
  sequence derived by mutation of obelin
MSSKYAVKLKTDFDNPRWIKRHKHMFDFLDINGNGKITLDEIVSKASDDICAKLEATPEQTKRHQVCVEAFFRGCGMEYGKEIAFPQFLDGWKQLATSELKKWARNEPTLIREWGDAVFDIFDKDGSGTITLDEWKAYGKISGISPSQEDCEATSRHCDLDNSGDLDVDEMTRQHLGFWYTLDPEADGLYGNGVP

SEQ ID NO: 105 - DNA, 588 bp, artificial sequence
  sequence derived by mutation of obelin
atgtcttcaaaatacgcagttaaactcaagactgactttgataatccacgatggatcaaaagacacaagcacatgtttgatttcctcgacatcaatggaaatggaaaaatcaccctcgatgaaattgtgtccaaggcatctgatgacatatgtgccaagctcgaagccacaccagaacaaacaaaacgccatcaagtttgtgttgaagctttctttagaggatgtggaatggaatatggtaaagaaattgccttcccacaattcctcgatggatggaaacaattggcgacttcagaactcaagaaatgggcaagaaacgaacctactctcattcgtgaatggggagatgctgtctttgatattttcgacaaagatggaagtggtacaatcactttggacgaatggaaagcttatggaaaaatctctggtatctctccatcacaagaagattgtgaagcgacatttcgacattgcggtttggacaacagtggtgaccttgatgttgacgagatgacaagacaacatcttggattctggtacactttggacccagaagctgatggtctctatggcaacggagttccctaa

SEQ ID NO: 106 - protein, 195 aa, artificial sequence
  sequence derived by mutation of obelin
MSSKYAVKLKTDFDNPRWIKRHKHMFDFLDINGNGKITLDEIVSKASDDICAKLEATPEQTKRHQVCVEAFFRGCGMEYGKEIAFPQFLDGWKQLATSELKKWARNEPTLIREWGDAVFDIFDKDGSGTITLDEWKAYGKISGISPSQEDCEATFRHCGLDNSGDLDVDEMTRQHLGFWYTLDPEADGLYGNGVP

SEQ ID NO: 107 - protein, 199 aa, artificial sequence
  sequence derived by mutation of aequorin
MLYDVPDYASLKLTSDFDNPRWIGRHRHMFNFLDVNHNGKISLDXMVYKASDIXINNLGATPEQAKRHKDAVEAFFGGAGMKYGVETDWPAYIEGWKKLATDELEKYAKNEPTLIRIWGDALFDIVXKDQNGAITLDXWKAYTKAAGIIQSSEDCEETXRVCXIDESGQLDVDEMTRQHLGFWYTMDPACEKLYGGAVP

SEQ ID NO: 108 - protein, 199 aa, artificial sequence
  sequence derived by mutation of aequorin
MLYDVPDYASLKLTSDFDNPRWIGRHKHMFNFLDVNHNGKISLDXMVYKASDIXINNLGATPEQAKRHKDAVEAFFGGAGMKYGVETDWPAYIEGWKKLATDELEKYAKNEPTLIRIWGDALFDIVXKDQNGAITLDXWKAYTKAAGIIQSSEDCEETXRVCXIDESGQLDVDEMTRRHLGFWYTMDPACEKLYGGAVP

SEQ ID NO: 109 - protein, 199 aa, artificial sequence
  sequence derived by mutation of aequorin
MLYDVPDYASLKLTSDFDNPRWIGRHKHMFNFLDVNHNGKISLDXMVYKASDIXINNLGATPEQAKRHKDAVEAFFGGAGMKYGVETDWPAYIEGWKKLATDELEKYAKNEPTLIRIWGDALFDIVXKDQNGAITLDXWKAYTKAAGIIQSSEDCEETXRVCXIDESGQLDVDEMTRQHIGFWYTMDPACEKLYGGAVP

SEQ ID NO: 110 - protein, 199 aa, artificial sequence
  sequence derived by mutation of aequorin
MLYDVPDYASLKLTSDFDNPRWIGRHRHMFNFLDVNHNGKISLDXMVYKASDIXINNLGATPEQAKRHKDAVEAFFGGAGMKYGVETDWPAYIEGWKKLATDELEKYAKNEPTLIRIWGDALFDIVXKDQNGAITLDXWKAYTKAAGIIQSSEDCEETXRVCXIDESGQLDVDEMTRRHLGFWYTMDPACEKLYGGAVP

SEQ ID NO: 111 - protein, 199 aa, artificial sequence
  sequence derived by mutation of aequorin
MLYDVPDYASLKLTSDFDNPRWIGRHRHMFNFLDVNHNGKISLDXMVYKASDIXINNLGATPEQAKRHKDAVEAFFGGAGMKYGVETDWPAYIEGWKKLATDELEKYAKNEPTLIRIWGDALFDIVXKDQNGAITLDXWKAYTKAAGIIQSSEDCEETXRVCXIDESGQLDVDEMTRQHIGFWYTMDPACEKLYGGAVP

SEQ ID NO: 112 - protein, 199 aa, artificial sequence
  sequence derived by mutation of aequorin
MLYDVPDYASLKLTSDFDNPRWIGRHKHMFNFLDVNHNGKISLDXMVYKASDIXINNLGATPEQAKRHKDAVEAFFGGAGMKYGVETDWPAYIEGWKKLATDELEKYAKNEPTLIRIWGDALFDIVXKDQNGAITLDXWKAYTKAAGIIQSSEDCEETXRVCXIDESGQLDVDEMTRRHIGFWYTMDPACEKLYGGAVP

SEQ ID NO: 113 - protein, 199 aa, artificial sequence
  sequence derived by mutation of aequorin
MLYDVPDYASLKLTSDFDNPRWIGRHRHMFNFLDVNHNGKISLDXMVYKASDIXINNLGATPEQAKRHKDAVEAFFGGAGMKYGVETDWPAYIEGWKKLATDELEKYAKNEPTLIRIWGDALFDIVXKDQNGAITLDXWKAYTKAAGIIQSSEDCEETXRVCXIDESGQLDVDEMTRRHIGFWYTMDPACEKLYGGAVP

SEQ ID NO: 114 - protein, 198 aa, artificial sequence
  sequence derived by mutation of clytin
MADTASKYAVKLRPNFDNPKWVNRHRFMFNFLDINGDGKITLDXIVSKASDDICAKLGATPEQTKRHQDAVEAFFKKIGMDYGKEVEFPAFVDGWKELANYDLKLWSQNKKSLIRDWGEAVFDIFXKDGSGSISLDXWKAYGRISGICSSDEDAEKTXKHCXLDNSGKLDVDEMTRQHLGFWYTLDPNADGLYGNFVP

SEQ ID NO: 115 - protein, 198 aa, artificial sequence
  sequence derived by mutation of clytin
MADTASKYAVKLRPNFDNPKWVNRHKFMFNFLDINGDGKITLDXIVSKASDDICAKLGATPEQTKRHQDAVEAFFKKIGMDYGKEVEFPAFVDGWKELANYDLKLWSQNKKSLIRDWGEAVFDIFXKDGSGSISLDXWKAYGRISGICSSDEDAEKTXKHCXLDNSGKLDVDEMTRRHLGFWYTLDPNADGLYGNFVP

SEQ ID NO: 116 - protein, 198 aa, artificial sequence
  sequence derived by mutation of clytin
MADTASKYAVKLRPNFDNPKWVNRHKFMFNFLDINGDGKITLDXIVSKASDDICAKLGATPEQTKRHQDAVEAFFKKIGMDYGKEVEFPAFVDGWKELANYDLKLWSQNKKSLIRDWGEAVFDIFXKDGSGSISLDXWKAYGRISGICSSDEDAEKTXKHCXLDNSGKLDVDEMTRQHIGFWYTLDPNADGLYGNFVP

SEQ ID NO: 117 - protein, 198 aa, artificial sequence
  sequence derived by mutation of clytin
MADTASKYAVKLRPNFDNPKWVNRHRFMFNFLDINGDGKITLDXIVSKASDDICAKLGATPEQTKRHQDAVEAFFKKIGMDYGKEVEFPAFVDGWKELANYDLKLWSQNKKSLIRDWGEAVFDIFXKDGSGSISLDXWKAYGRISGICSSDEDAEKTXKHCXLDNSGKLDVDEMTRRHLGFWYTLDPNADGLYGNFVP

SEQ ID NO: 118 - protein, 198 aa, artificial sequence
  sequence derived by mutation of clytin
MADTASKYAVKLRPNFDNPKWVNRHRFMFNFLDINGDGKITLDXIVSKASDDICAKLGATPEQTKRHQDAVEAFFKKIGMDYGKEVEFPAFVDGWKELANYDLKLWSQNKKSLIRDWGEAVFDIFXKDGSGSISLDXWKAYGRISGICSSDEDAEKTXKHCXLDNSGKLDVDEMTRQHIGFWYTLDPNADGLYGNFVP

SEQ ID NO: 119 - protein, 198 aa, artificial sequence
  sequence derived by mutation of clytin
MADTASKYAVKLRPNFDNPKWVNRHKFMFNFLDINGDGKITLDXIVSKASDDICAKLGATPEQTKRHQDAVEAFFKKIGMDYGKEVEFPAFVDGWKELANYDLKLWSQNKKSLIRDWGEAVFDIFXKDGSGSISLDXWKAYGRISGICSSDEDAEKTXKHCXLDNSGKLDVDEMTRRHIGFWYTLDPNADGLYGNFVP

SEQ ID NO: 120 - protein, 198 aa, artificial sequence
  sequence derived by mutation of clytin
MADTASKYAVKLRPNFDNPKWVNRHRFMFNFLDINGDGKITLDXIVSKASDDICAKLGATPEQTKRHQDAVEAFFKKIGMDYGKEVEFPAFVDGWKELANYDLKLWSQNKKSLIRDWGEAVFDIFXKDGSGSISLDXWKAYGRISGICSSDEDAEKTXKHCXLDNSGKLDVDEMTRRHIGFWYTLDPNADGLYGNFVP

SEQ ID NO: 121 - protein, 198 aa, artificial sequence
  sequence derived by mutation of mitrocomin
MSMGSRYAVKLTTDFDNPKWIARHRHMFNFLDINSNGQINLNXMVHKASNIICKKLGATEEQTKRHQKCVEDFFGGAGLEYDKDTTWPEYIEGWKRLAKTELERHSKNQVTLIRLWGDALFDIIXKDRNGSVSLDXWIQYTHCAGIQQSRGQCEATXAHCXLDGDGKLDVDEMTRQHLGFWYSVDPTCEGLYGGAVPY

SEQ ID NO: 122 - protein, 198 aa, artificial sequence
  sequence derived by mutation of mitrocomin
MSMGSRYAVKLTTDFDNPKWIARHKHMFNFLDINSNGQINLNXMVHKASNIICKKLGATEEQTKRHQKCVEDFFGGAGLEYDKDTTWPEYIEGWKRLAKTELERHSKNQVTLIRLWGDALFDIIXKDRNGSVSLDXWIQYTHCAGIQQSRGQCEATXAHCXLDGDGKLDVDEMTRRHLGFWYSVDPTCEGLYGGAVPY

SEQ ID NO: 123 - protein, 198 aa, artificial sequence
  sequence derived by mutation of mitrocomin
MSMGSRYAVKLTTDFDNPKWIARHKHMFNFLDINSNGQINLNXMVHKASNIICKKLGATEEQTKRHQKCVEDFFGGAGLEYDKDTTWPEYIEGWKRLAKTELERHSKNQVTLIRLWGDALFDIIXKDRNGSVSLDXWIQYTHCAGIQQSRGQCEATXAHCXLDGDGKLDVDEMTRQHIGFWYSVDPTCEGLYGGAVPY

SEQ ID NO: 124 - protein, 198 aa, artificial sequence
  sequence derived by mutation of mitrocomin
MSMGSRYAVKLTTDFDNPKWIARHRHMFNFLDINSNGQINLNXMVHKASNIICKKLGATEEQTKRHQKCVEDFFGGAGLEYDKDTTWPEYIEGWKRLAKTELERHSKNQVTLIRLWGDALFDIIXKDRNGSVSLDXWIQYTHCAGIQQSRGQCEATXAHCXLDGDGKLDVDEMTRRHLGFWYSVDPTCEGLYGGAVPY

SEQ ID NO: 125 - protein, 198 aa, artificial sequence
  sequence derived by mutation of mitrocomin
MSMGSRYAVKLTTDFDNPKWIARHRHMFNFLDINSNGQINLNXMVHKASNIICKKLGATEEQTKRHQKCVEDFFGGAGLEYDKDTTWPEYIEGWKRLAKTELERHSKNQVTLIRLWGDALFDIIXKDRNGSVSLDXWIQYTHCAGIQQSRGQCEATXAHCXLDGDGKLDVDEMTRQHIGFWYSVDPTCEGLYGGAVPY

SEQ ID NO: 126 - protein, 198 aa, artificial sequence
  sequence derived by mutation of mitrocomin
MSMGSRYAVKLTTDFDNPKWIARHKHMFNFLDINSNGQINLNXMVHKASNIICKKLGATEEQTKRHQKCVEDFFGGAGLEYDKDTTWPEYIEGWKRLAKTELERHSKNQVTLIRLWGDALFDIIXKDRNGSVSLDXWIQYTHCAGIQQSRGQCEATXAHCXLDGDGKLDVDEMTRRHIGFWYSVDPTCEGLYGGAVPY

SEQ ID NO: 127 - protein, 198 aa, artificial sequence
  sequence derived by mutation of mitrocomin
MSMGSRYAVKLTTDFDNPKWIARHRHMFNFLDINSNGQINLNXMVHKASNIICKKLGATEEQTKRHQKCVEDFFGGAGLEYDKDTTWPEYIEGWKRLAKTELERHSKNQVTLIRLWGDALFDIIXKDRNGSVSLDXWIQYTHCAGIQQSRGQCEATXAHCXLDGDGKLDVDEMTRRHIGFWYSVDPTCEGLYGGAVPY

SEQ ID NO: 128 - protein, 195 aa, artificial sequence
  sequence derived by mutation of obelin
MSSKYAVKLKTDFDNPRWIKRHRHMFDFLDINGNGKITLDXIVSKASDDICAKLEATPEQTKRHQVCVEAFFRGCGMEYGKEIAFPQFLDGWKQLATSELKKWARNEPTLIREWGDAVFDIFXKDGSGTITLDXWKAYGKISGISPSQEDCEATXRHCXLDNSGDLDVDEMTRQHLGFWYTLDPEADGLYGNGVP

SEQ ID NO: 129 - protein, 195 aa, artificial sequence
  sequence derived by mutation of obelin
MSSKYAVKLKTDFDNPRWIKRHKHMFDFLDINGNGKITLDXIVSKASDDICAKLEATPEQTKRHQVCVEAFFRGCGMEYGKEIAFPQFLDGWKQLATSELKKWARNEPTLIREWGDAVFDIFXKDGSGTITLDXWKAYGKISGISPSQEDCEATXRHCXLDNSGDLDVDEMTRRHLGFWYTLDPEADGLYGNGVP

SEQ ID NO: 130 - protein, 195 aa, artificial sequence
  sequence derived by mutation of obelin
MSSKYAVKLKTDFDNPRWIKRHKHMFDFLDINGNGKITLDXIVSKASDDICAKLEATPEQTKRHQVCVEAFFRGCGMEYGKEIAFPQFLDGWKQLATSELKKWARNEPTLIREWGDAVFDIFXKDGSGTITLDXWKAYGKISGISPSQEDCEATXRHCXLDNSGDLDVDEMTRQHIGFWYTLDPEADGLYGNGVP

SEQ ID NO: 131 - protein, 195 aa, artificial sequence
  sequence derived by mutation of obelin
MSSKYAVKLKTDFDNPRWIKRHRHMFDFLDINGNGKITLDXIVSKASDDICAKLEATPEQTKRHQVCVEAFFRGCGMEYGKEIAFPQFLDGWKQLATSELKKWARNEPTLIREWGDAVFDIFXKDGSGTITLDXWKAYGKISGISPSQEDCEATXRHCXLDNSGDLDVDEMTRRHLGFWYTLDPEADGLYGNGVP

SEQ ID NO: 132 - protein, 195 aa, artificial sequence
  sequence derived by mutation of obelin
MSSKYAVKLKTDFDNPRWIKRHRHMFDFLDINGNGKITLDXIVSKASDDICAKLEATPEQTKRHQVCVEAFFRGCGMEYGKEIAFPQFLDGWKQLATSELKKWARNEPTLIREWGDAVFDIFXKDGSGTITLDXWKAYGKISGISPSQEDCEATXRHCXLDNSGDLDVDEMTRQHIGFWYTLDPEADGLYGNGVP

SEQ ID NO: 133 - protein, 195 aa, artificial sequence
  sequence derived by mutation of obelin
MSSKYAVKLKTDFDNPRWIKRHKHMFDFLDINGNGKITLDXIVSKASDDICAKLEATPEQTKRHQVCVEAFFRGCGMEYGKEIAFPQFLDGWKQLATSELKKWARNEPTLIREWGDAVFDIFXKDGSGTITLDXWKAYGKISGISPSQEDCEATXRHCXLDNSGDLDVDEMTRRHIGFWYTLDPEADGLYGNGVP

SEQ ID NO: 134 - protein, 195 aa, artificial sequence
  sequence derived by mutation of obelin
MSSKYAVKLKTDFDNPRWIKRHRHMFDFLDINGNGKITLDXIVSKASDDICAKLEATPEQTKRHQVCVEAFFRGCGMEYGKEIAFPQFLDGWKQLATSELKKWARNEPTLIREWGDAVFDIFXKDGSGTITLDXWKAYGKISGISPSQEDCEATXRHCXLDNSGDLDVDEMTRRHIGFWYTLDPEADGLYGNGVP

(19) United States
(12) Patent Application Publication
(10) Pub. No.: US 2005/0130262 A1
(43) Pub. Date: Jun. 16, 2005

(54) Mutated photoproteins and their uses

(76) Inventors: Bertrand Lambolez, Paris (FR); Nathalie Gibelin, Chatenay Malabry (FR); Gaelle Bourout, Paris (FR); Ludovic Eric Tricoire, Montrouge (FR); Olivier Arsene Courjean, Paris (FR); Keisuke Tsuzuki, Gunma (JP); Jean Rossier, Paris (FR)

Correspondence Address: YOUNG & THOMPSON, 745 SOUTH 23RD STREET, 2ND FLOOR, ARLINGTON, VA, 22202, US

(21) Appl. No.: 10/484,380

(22) PCT Filed: Jul. 12, 2002
(86) PCT No.: PCT/FR02/02492

(30) Foreign Application Priority Data
Jul. 12, 2001 (FR) 01/09293

(51) Int. Cl. C07K 14/435; C07H 21/04; C12N 5/02

(52) U.S. Cl. 435/69.1; 435/320.1; 435/325; 530/350; 536/23.5

(57) ABSTRACT

Mutated photoproteins derived from isolated jellyfish photoproteins, characterised in that they exhibit a thermostability higher than that of the photoproteins from which they are derived, and are called thermostable mutated photoproteins, and/or a luminescence duration longer than that of the photoproteins from which they are derived, and are called persistent mutated photoproteins. The invention also concerns the use of said mutated photoproteins in methods for detecting in vitro molecules in a biological sample, methods for detecting compounds with enzymatic activity in a biological sample, or methods for detecting intracellular calcium variations.

A subject of the present invention is mutated photoproteins derived from the photoproteins isolated from jellyfish, said mutated photoproteins being characterized by a thermostability, and/or by a luminescence time greater than those of the photoproteins from which they derive, as well as the use of these proteins, in particular within the context of the implementation of processes for in vitro detection of molecules, processes for detecting compounds with enzymatic activity in a biological sample, or processes for detecting variations in intracellular calcium induced by various agents.

Aequorin is a photoprotein of the jellyfish Aequoria Victoria which is constituted by a protein part called apoequorin and a prosthetic group, coelenterazine. This photoprotein has the property of emitting light when it is in the presence of calcium ions (Ca²⁺). This property makes it possible in particular to detect calcium variations in the cells. This photoprotein is also used as a marker in order to detect small quantities of organic products (down to less than a hundred molecules) by virtue of an extremely high ratio of signal to background noise.

Thus, aequorin is beginning to be used in commercial systems for detecting molecules. Its use offers several advantages: a very wide dynamic range which makes it possible to detect and quantify the molecules over several orders of magnitude, and an extremely low background noise which makes it possible to detect the presence of only a few tens of molecules in a sample. This makes it possible to avoid resorting to amplification techniques, such as PCR for the detection of nucleic acids.

However, the natural photoproteins, and aequorin in particular are very sensitive to temperature variations which can denature their power to emit light.

Moreover, the emission kinetics of the natural photoprotein is extremely rapid (of the order of a second) and requires a “sample by sample” analysis in a rapid-injection luminometer. This poses a problem for using the aequorin in a high throughput screening system which requires the simultaneous analysis of a large number of samples (HTS, High Throughput Screening).

The present invention results from the demonstration by the inventors of the fact that certain mutations in the peptide sequence of aequorin make it possible to obtain aequorin mutants which are much less sensitive to temperature (at 37° C., the mutant photoproteins lose their light-emitting properties in a few days instead of a few hours for the natural protein), as well as mutants having very slowed-down light-emission kinetics (from ten seconds to a minute) which make it possible to analyze the samples simultaneously (multi-well micro-plate format).

Thus a principal object of the present invention is to provide novel photoproteins which are less sensitive to temperature rises, and the transport and the storage of which are facilitated due to their better stability.

Another object of the present invention is to provide novel photoproteins resistant up to 50° C., which simplifies their use, in particular in nucleic acid detection experiments.

Another object of the invention is to provide novel photoproteins the luminescence time of which is distinctly greater than that of the photoproteins from which they derive, which makes it possible to use them within the context of high throughput screening, in particular for the in vitro detection of trace organic molecules.

Another object of the invention is to provide kits comprising these novel photoproteins, for the implementation of measurement and detection processes as mentioned above.

A principal object of the invention is the use of photoproteins isolated from jellyfish for the preparation of mutated photoproteins having a thermostability greater than that of the photoproteins from which they derive, also designated thermostable mutated photoproteins, and/or by a luminescence time greater than that of the photoproteins from which they derive, also designated persistent mutated photoproteins, said mutated photoproteins being characterized in that their stability over time is increased at 37° C. by a factor of at least approximately 10, and/or in that their luminescence time is increased by a factor of at least approximately 10, in comparison with the photoproteins from which they derive.

It should be stressed that by the expression mutated photoprotein above and below, is meant any photoprotein made up of a protein part derived by mutation of the protein part of the jellyfish photoprotein from which it originates, and of a prosthetic group, such as coelenterazine.

A subject of the invention is also a process for preparing thermostable and/or persistent mutated photoproteins, said mutated photoproteins being characterized in that their stability over time is increased at 37° C. by a factor of at least approximately 10, and/or in that their luminescence time is increased by a factor of at least approximately 10, in comparison with the jellyfish photoproteins from which they derive, characterized in that it comprises the implementation of one or more mutations of said jellyfish photoproteins, said mutations being chosen from:
at least one of the three mutations increasing the thermostability of said photoproteins, and chosen from the following:
suppression of the lysine (K) contained in the unit RHKX₁MFX₂ in which X₁=H or F, and X₂=N or D, this peptide unit being preserved in said photoproteins, or substitution of this lysine by a natural or non-natural amino acid, in particular substitution of this lysine by an arginine,
suppression of the glutamine (Q) contained in the unit DEMTRQHLGFWY, this peptide unit being preserved in said photoproteins, or substitution of this glutamine by a natural or non-natural amino acid, in particular substitution of this glutamine by an arginine,
suppression of the leucine (L) contained in the abovementioned unit DEMTRQHLGFWY, or substitution of this leucine by a natural or non-natural amino acid, in particular substitution of this leucine by an isoleucine,
and/or at least one of the six mutations increasing the luminescence time of said photoproteins, and chosen from the following:
suppression of the glutamate (E) contained in the unit DX₁NX₂X₃GX₄IX₅LX₆E in which X₁=V or I, X₂=H, G or S, X₃=N or D, X₄=K or Q, X₅=S, T or N, and X₆=D or N, this peptide unit being preserved in said photoproteins, or substitution of this glutamate by a natural or non-natural amino acid, in particular substitution of this glutamate by a small amino acid such as glycine, alanine, cysteine, or threonine,
suppression of the first aspartate (D) contained in the unit DKDX₁X₂GX₃X₄X₅LDE in which X₁=Q, G or R, X₂=N or S, X₃=A, S or T, X₄=I or V, and X₅=T or S, this peptide unit being preserved in said photoproteins, or substitution of this aspartate by a natural or non-natural amino acid, in particular substitution of this aspartate by a small amino acid such as glycine, alanine, cysteine, or threonine,
suppression of the glutamate (E) contained in the abovementioned unit DKDX₁X₂GX₃X₄X₅LDE, this peptide unit being preserved in said photoproteins, or substitution of this glutamate by a natural or non-natural amino acid, in particular substitution of this glutamate by a small amino acid such as glycine, alanine, cysteine, or threonine,
suppression of the phenylalanine (F) contained in the unit EX₁TFX₂X₃ in which X₁=E, K or A, X₂=R, K or A, and X₃=V or H, this peptide unit being preserved in said photoproteins, or substitution of this phenylalanine by a natural or non-natural amino acid, in particular substitution of this phenylalanine by a serine, or by a small amino acid such as glycine, alanine, cysteine, or threonine,
suppression of the first aspartate (D) contained in the unit DX₁DX₂X₃GX₄LDVDE, in which X₁=I or L, X₂=E, N or G, X₃=S or D, and X₄=Q, K or D, this peptide unit being preserved in said photoproteins, or substitution of this aspartate by a natural or non-natural amino acid, in particular substitution of this aspartate by a small amino acid such as glycine, alanine, cysteine, or threonine,
suppression of the valine in position 54 of the peptide sequence SEQ ID NO: 2 of the aequorin, or substitution of this valine by a natural or non-natural amino acid, in particular substitution of this valine by an alanine, or by a small amino acid such as glycine, alanine, cysteine, or threonine.

The invention also relates to the use of the thermostable and/or persistent mutated photoproteins for the implementation of:
processes for in vitro detection of molecules, such as proteins or antigens or nucleic acids in a biological sample, in particular within the context of in vitro screening for bacteria such as Listeria in food, or within the context of screening for pathogenic agents such as the HIV virus in humans,
processes for detecting compounds with enzymatic activity in a biological sample, in particular within the context of screening for molecules activating or inhibiting a specific enzymatic activity,
processes for detecting the intracellular calcium variations induced by various agents, in particular within the context of screening for molecules acting on a nucleic or protein sequence fused to the mutated photoprotein, or coexpressed with the mutated photoprotein in the abovementioned host cells,
in which the temperatures used can reach more than 50° C., and/or the reading time after activation of the photoprotein can be greater than 5 minutes, which considerably limits the reading noises.

A subject of the invention is also the mutated photoproteins derived from the photoproteins isolated from jellyfish, said mutated photoproteins being characterized by a thermostability greater than that of the photoproteins from which they derive, and are designated thermostable mutated photoproteins, and/or by a luminescence time greater than that of the photoproteins from which they derive, and are designated persistent mutated photoproteins.

Advantageously, the mutated photoproteins according to the invention, are such that the stability over time is increased at 37° C. by a factor of at least approximately 10, and/or their luminescence time is increased by a factor of at least approximately 10, in comparison with the photoproteins from which they derive.

Advantageously also, the mutated photoproteins according to the invention, are characterized in that they are stable for at least approximately 30 minutes up to a temperature of approximately 50° C., and in that they can be kept for at least approximately 4 days at temperatures which can reach up to 37° C., and/or in that their luminescence time is comprised between approximately 1 minute and approximately 5 minutes.

A more particular subject of the invention is the mutated photoproteins as defined above, characterized in that they comprise:
at least one of the three mutations increasing their thermostability, chosen from the following:
suppression of the lysine (K) contained in the unit RHKX₁MFX₂ in which X₁=H or F, and X₂=N or D, this peptide unit being preserved in said photoproteins, or substitution of this lysine by a natural or non-natural amino acid, in particular substitution of this lysine by an arginine,
suppression of the glutamine (Q) contained in the unit DEMTRQHLGFWY, this peptide unit being preserved in said photoproteins, or substitution of this glutamine by a natural or non-natural amino acid, in particular substitution of this glutamine by an arginine,
suppression of the leucine (L) contained in the abovementioned unit DEMTRQHLGFWY, or substitution of this leucine by a natural or non-natural amino acid, in particular substitution of this leucine by an isoleucine,
and/or at least one of the six mutations increasing their luminescence time, chosen from the following:
suppression of the glutamate (E) contained in the unit DX₁NX₂X₃GX₄IX₅LX₆E in which X₁=V or I, X₂=H, G or S, X₃=N or D, X₄=K or Q, X₅=S, T or N, and X₆=D or N, this peptide unit being preserved in said photoproteins, or substitution of this glutamate by a natural or non-natural amino acid, excluding serine, in particular substitution of this glutamate by a small amino acid such as glycine, alanine, cysteine, or threonine,
suppression of the first aspartate (D) contained in the unit DKDX₁X₂GX₃X₄X₅LDE in which X₁=Q, G or R, X₂=N or S, X₃=A, S or T, X₄=I or V, and X₅=T or S, this peptide unit being preserved in said photoproteins, or substitution of this aspartate by a natural or non-natural amino acid, excluding serine, in particular substitution of this aspartate by a small amino acid such as glycine, alanine, cysteine, or threonine,
suppression of the glutamate (E) contained in the abovementioned unit DKDX₁X₂GX₃X₄X₅LDE, this peptide unit being preserved in said photoproteins, or substitution of this glutamate by a natural or non-natural amino acid, excluding serine, in particular substitution of this glutamate by a small amino acid such as glycine, alanine, cysteine, or threonine,
suppression of the phenylalanine (F) contained in the unit EX₁TFX₂X₃ in which X₁=E, K or A, X₂=R, K or A, and X₃=V or H, this peptide unit being preserved in said photoproteins, or substitution of this phenylalanine by a natural or non-natural amino acid, in particular substitution of this phenylalanine by a serine, or by a small amino acid such as glycine, alanine, cysteine, or threonine,
suppression of the first aspartate (D) contained in the unit DX₁DX₂X₃GX₄LDVDE, in which X₁=I or L, X₂=E, N or G, X₃=S or D, and X₄=Q, K or D, this peptide unit being preserved in said photoproteins, or substitution of this aspartate by a natural or non-natural amino acid, excluding serine, in particular substitution of this aspartate by a small amino acid such as glycine, alanine, cysteine, or threonine,
suppression of the valine in position 54 of the peptide sequence SEQ ID NO: 2 of aequorin, or substitution of this valine by a natural or non-natural amino acid, in particular substitution of this valine by an alanine, or by a small amino acid such as glycine, alanine, cysteine, or threonine.

Thermostable mutated photoproteins which are particularly preferred according to the invention, are characterized in that they comprise at least one of the following two mutations:
suppression of the glutamine contained in the unit DEMTRQHLGFWY, this peptide unit being preserved in said photoproteins, or substitution of this glutamine by a natural or non-natural amino acid, in particular substitution of this glutamine by an arginine,
suppression of the leucine contained in the abovementioned unit DEMTRQHLGFWY, or substitution of this leucine by a natural or non-natural amino acid, in particular substitution of this leucine by an isoleucine.

Persistent mutated photoproteins which are particularly preferred according to the invention, are characterized in that they comprise at least one of the following mutations:
suppression of the first aspartate (D) contained in the unit DKDX₁X₂GX₃X₄X₅LDE in which X₁=Q, G or R, X₂=N or S, X₃=A, S or T, X₄=I or V, and X₅=T or S, this peptide unit being preserved in said photoproteins, or substitution of this aspartate by a natural or non-natural amino acid, excluding serine, in particular substitution of this aspartate by a small amino acid such as glycine, alanine, cysteine, or threonine,
suppression of the glutamate (E) contained in the abovementioned unit DKDX₁X₂GX₃X₄X₅LDE, this peptide unit being preserved in said photoproteins, or substitution of this glutamate by a natural or non-natural amino acid, excluding serine, in particular substitution of this glutamate by a small amino acid such as glycine, alanine, cysteine, or threonine,
suppression of the first aspartate (D) contained in the unit DX₁DX₂X₃GX₄LDVDE, in which X₁=I or L, X₂=E, N or G, X₃=S or D, and X₄=Q, K or D, this peptide unit being preserved in said photoproteins, or substitution of this aspartate by a natural or non-natural amino acid, excluding serine, in particular substitution of this aspartate by a small amino acid such as glycine, alanine, cysteine, or threonine.

A more particular subject of the invention is the mutated photoproteins as defined above, characterized in that they derive from:
aequorin extracted from the jellyfish Aequoria Victoria, said aequorin being as represented by the sequence SEQ ID NO: 2,
clytin extracted from the jellyfish Clytia gregaria, said clytin being as represented by the sequence SEQ ID NO: 18,
mitrocomin extracted from the jellyfish Mitrocoma cellularia, said mitrocomin being such as represented by the sequence SEQ ID NO: 34,
or obelin extracted from the jellyfish Obelia longissima, said obelin being as represented by the sequence SEQ ID NO: 50.

The invention relates more particularly to the thermostable mutated photoproteins derived from aequorin as defined above, and chosen from the proteins comprising the following sequences:
the peptide sequence SEQ ID NO: 4, corresponding to the sequence SEQ ID NO: 2 in which the lysine in position 27 is replaced by an arginine,
the peptide sequence SEQ ID NO: 6, corresponding to the sequence SEQ ID NO: 2 in which the glutamine in position 178 is replaced by an arginine,
the peptide sequence SEQ ID NO: 8, corresponding to the sequence SEQ ID NO: 2 in which the leucine in position 180 is replaced by an isoleucine,
the peptide sequence SEQ ID NO: 10, corresponding to the sequence SEQ ID NO: 2 in which the lysine in position 27 is replaced by an arginine, and the glutamine in position 178 is replaced by an arginine,
the peptide sequence SEQ ID NO: 12, corresponding to the sequence SEQ ID NO: 2 in which the lysine in position 27 is replaced by an arginine, and the leucine in position 180 is replaced by an isoleucine,
the peptide sequence SEQ ID NO: 14, corresponding to the sequence SEQ ID NO: 2 in which the glutamine in position 178 is replaced by an arginine, and the leucine in position 180 is replaced by an isoleucine,
the peptide sequence SEQ ID NO: 16, corresponding to the sequence SEQ ID NO: 2 in which the lysine in position 27 is replaced by an arginine, the glutamine in position 178 is replaced by an arginine, and the leucine in position 180 is replaced by an isoleucine.

A particular subject of the invention is the thermostable mutated photoproteins derived from clytin as defined above, chosen from the proteins comprising the following sequences:
the peptide sequence SEQ ID NO: 20, corresponding to the sequence SEQ ID NO: 18 in which the lysine in position 26 is replaced by an arginine,
the peptide sequence SEQ ID NO: 22, corresponding to the sequence SEQ ID NO: 18 in which the glutamine in position 177 is replaced by an arginine,
the peptide sequence SEQ ID NO: 24, corresponding to the sequence SEQ ID NO: 18 in which the leucine in position 179 is replaced by an isoleucine,
the peptide sequence SEQ ID NO: 26, corresponding to the sequence SEQ ID NO: 18 in which the lysine in position 26 is replaced by an arginine, and the glutamine in position 177 is replaced by an arginine,
the peptide sequence SEQ ID NO: 28, corresponding to the sequence SEQ ID NO: 18 in which the lysine in position 26 is replaced by an arginine, and the leucine in position 179 is replaced by an isoleucine,
the peptide sequence SEQ ID NO: 30, corresponding to the sequence SEQ ID NO: 18 in which the glutamine in position 177 is replaced by an arginine, and the leucine in position 179 is replaced by an isoleucine,
the peptide sequence SEQ ID NO: 32, corresponding to the sequence SEQ ID NO: 18 in which the lysine in position 26 is replaced by an arginine, the glutamine in position 177 is replaced by an arginine, and the leucine in position 179 is replaced by an isoleucine.

The invention relates more particularly to the thermostable mutated photoproteins derived from mitrocomin as defined above, chosen from the proteins comprising the following sequences:
the peptide sequence SEQ ID NO: 36, corresponding to the sequence SEQ ID NO: 34 in which the lysine in position 25 is replaced by an arginine,
the peptide sequence SEQ ID NO: 38, corresponding to the sequence SEQ ID NO: 34 in which the glutamine in position 176 is replaced by an arginine,
the peptide sequence SEQ ID NO: 40, corresponding to the sequence SEQ ID NO: 34 in which the leucine in position 178 is replaced by an isoleucine,
the peptide sequence SEQ ID NO: 42, corresponding to the sequence SEQ ID NO: 34 in which the lysine in position 25 is replaced by an arginine, and the glutamine in position 176 is replaced by an arginine,
the peptide sequence SEQ ID NO: 44 corresponding to the sequence SEQ ID NO: 34 in which the lysine in position 25 is replaced by an arginine, and the leucine in position 178 is replaced by an isoleucine,
the peptide sequence SEQ ID NO: 46, corresponding to the sequence SEQ ID NO: 34 in which the glutamine in position 176 is replaced by an arginine, and the leucine in position 178 is replaced by an isoleucine,
the peptide sequence SEQ ID NO: 48, corresponding to the sequence SEQ ID NO: 34 in which the lysine in position 25 is replaced by an arginine, the glutamine in position 176 is replaced by an arginine, and the leucine in position 178 is replaced by an isoleucine.

The invention relates more particularly to the thermostable mutated photoproteins derived from obelin as defined above, chosen from the proteins comprising the following sequences:
the peptide sequence SEQ ID NO: 52, corresponding to the sequence SEQ ID NO: 50 in which the lysine in position 23 is replaced by an arginine,
the peptide sequence SEQ ID NO: 54, corresponding to the sequence SEQ ID NO: 50 in which the glutamine in position 174 is replaced by an arginine,
the peptide sequence SEQ ID NO: 56, corresponding to the sequence SEQ ID NO: 50 in which the leucine in position 176 is replaced by an isoleucine,
the peptide sequence SEQ ID NO: 58, corresponding to the sequence SEQ ID NO: 50 in which the lysine in position 23 is replaced by an arginine, and the glutamine in position 174 is replaced by an arginine,
the peptide sequence SEQ ID NO: 60, corresponding to the sequence SEQ ID NO: 50 in which the lysine in position 23 is replaced by an arginine, and the leucine in position 176 is replaced by an isoleucine,
the peptide sequence SEQ ID NO: 62 corresponding to the sequence SEQ ID NO: 50 in which the glutamine in position 174 is replaced by an arginine, and the leucine in position 176 is replaced by an isoleucine,
the peptide sequence SEQ ID NO: 64, corresponding to the sequence SEQ ID NO: 50 in which the lysine in position 23 is replaced by an arginine, the glutamine in position 174 is replaced by an arginine, and the leucine in position 176 is replaced by an isoleucine.

The invention relates more particularly to the persistent mutated photoproteins derived from aequorin as defined above, and chosen from the proteins comprising the following sequences:
the peptide sequence SEQ ID NO: 66 corresponding to the sequence SEQ ID NO: 2 in which the glutamate in position 45 is replaced by a glycine,
the peptide sequence SEQ ID NO: 68 corresponding to the sequence SEQ ID NO: 2 in which the valine in position 54 is replaced by an alanine,
the peptide sequence SEQ ID NO: 70 corresponding to the sequence SEQ ID NO: 2 in which the aspartate in position 127 is replaced by a glycine,
the peptide sequence SEQ ID NO: 72 corresponding to the sequence SEQ ID NO: 2 in which the glutamate in position 138 is replaced by a glycine,
the peptide sequence SEQ ID NO: 74 corresponding to the sequence SEQ ID NO: 2 in which the phenylalanine in position 159 is replaced by a serine,
the peptide sequence SEQ ID NO: 76 corresponding to the sequence SEQ ID NO: 2 in which the aspartate in position 163 is replaced by a glycine.

A particular subject of the invention is the persistent mutated photoproteins derived from clytin as defined above, chosen from the proteins comprising the following sequences:
the peptide sequence SEQ ID NO: 78 corresponding to the sequence SEQ ID NO: 18 in which the glutamate in position 44 is replaced by a glycine,
the peptide sequence SEQ ID NO: 80 corresponding to the sequence SEQ ID NO: 18 in which the aspartate in position 126 is replaced by a glycine,
the peptide sequence SEQ ID NO: 82 corresponding to the sequence SEQ ID NO: 18 in which the glutamate in position 137 is replaced by a glycine,
the peptide sequence SEQ ID NO: 84 corresponding to the sequence SEQ ID NO: 18 in which the phenylalanine in position 158 is replaced by a serine,
the peptide sequence SEQ ID NO: 86 corresponding to the sequence SEQ ID NO: 18 in which the aspartate in position 162 is replaced by a glycine.

The invention relates more particularly to the persistent mutated photoproteins derived from mitrocomin as defined above, chosen from the proteins comprising the following sequences:
the peptide sequence SEQ ID NO: 88 corresponding to the sequence SEQ ID NO: 34 in which the glutamate in position 43 is replaced by a glycine,
the peptide sequence SEQ ID NO: 90 corresponding to the sequence SEQ ID NO: 34 in which the aspartate in position 125 is replaced by a glycine,
the peptide sequence SEQ ID NO: 92 corresponding to the sequence SEQ ID NO: 34 in which the glutamate in position 136 is replaced by a glycine,
the peptide sequence SEQ ID NO: 94 corresponding to the sequence SEQ ID NO: 34 in which the phenylalanine in position 157 is replaced by a serine,
the peptide sequence SEQ ID NO: 96 corresponding to the sequence SEQ ID NO: 34 in which the aspartate in position 161 is replaced by a glycine.

The invention relates more particularly to the persistent mutated photoproteins derived from obelin as defined above, chosen from the proteins comprising the following sequences:
the peptide sequence SEQ ID NO: 98 corresponding to the sequence SEQ ID NO: 50 in which the glutamate in position 41 is replaced by a glycine,
the peptide sequence SEQ ID NO: 100 corresponding to the sequence SEQ ID NO: 50 in which the aspartate in position 123 is replaced by a glycine,
the peptide sequence SEQ ID NO: 102 corresponding to the sequence SEQ ID NO: 50 in which the glutamate in position 134 is replaced by a glycine,
the peptide sequence SEQ ID NO: 104 corresponding to the sequence SEQ ID NO: 50 in which the phenylalanine in position 155 is replaced by a serine,
the peptide sequence SEQ ID NO: 106 corresponding to the sequence SEQ ID NO: 50 in which the as partate in position 159 is replaced by a glycine.

A more particular subject of the invention is the thermostable and persistent mutated photoproteins as defined above, chosen from:
the proteins derived from aequorin of sequences SEQ ID NO: 107, 108, 109, 110, 111, 112, 113, and corresponding to the sequences SEQ ID NO: 4, 6, 8, 10, 12, 14, 16, in which E in position 45 is replaced by G, and/or V in position 54 is replaced by A, and/or D in position 127 is replaced by G, and/or E in position 138 is replaced by G, and/or F in position 159 is replaced by S, and/or D in position 163 is replaced by G,
the proteins derived from clytin of sequences SEQ ID NO: 114, 115, 116, 117, 118, 119, 120, and corresponding to the following sequences: SEQ ID NO: 20, 22, 24, 26, 28, 30, 32, in which E in position 44 is replaced by G, and/or D in position 126 is replaced by G, and/or E in position 137 is replaced by G, and/or F in position 158 is replaced by S, and/or D in position 162 is replaced by G,
the proteins of mitrocomin of sequences SEQ ID NO: 121, 122, 123, 124, 125, 126, 127, and corresponding to the following sequences: SEQ ID NO: 36, 38, 40, 42, 44, 46, 48, in which E in position 43 is replaced by G, and/or D in position 125 is replaced by G, and/or E in position 136 is replaced by G, and/or F in position 157 is replaced by S, and/or D in position 161 is replaced by G,
the proteins derived from obelin of sequences SEQ ID NO: 128, 129, 130, 131, 132, 133, 134, and corresponding to the following sequences: SEQ ID NO: 52, 54, 56, 58, 60, 62, 64, in which E in position 41 is replaced by G, and/or D in position 123 is replaced by G, and/or E in position 134 is replaced by G, and/or F in position 155 is replaced by S, and/or D in position 159 is replaced by G.

A more particular subject of the invention is the mutated photoproteins as defined above, characterized in that they are bound:
to a protein or nucleic probe capable of recognizing specific antigens or proteins or nucleic acids,
or to a specific substrate with a specific enzymatic activity,
or to a molecule capable of forming a complex with another molecule, such as the avidin-biotin complex.

The invention also relates to the nucleotide sequences coding for the mutated photoproteins defined above.

A more particular subject of the invention is therefore the above-mentioned nucleotide sequences, coding for the mutated photoproteins as defined above, chosen from the nucleic acids comprising the sequences SEQ ID NO: 3, 5, 7, 9, 11, 13, 15, 19, 21, 23, 25, 27, 29, 31, 35, 37, 39, 41, 43, 45, 47, 51, 53, 55, 57, 59, 61, 63, 65, 67, 69, 71, 73, 75, 77, 79, 81, 83, 85, 87, 89, 91, 93, 95, 97, 99, 101, 103, 105, coding respectively for the sequences SEQ ID NO: 4, 6, 8, 10, 12, 14, 16, 20, 22, 24, 26, 28, 30, 32, 36, 38, 40, 42, 44, 46, 48, 52, 54, 56, 58, 60, 62, 64, 66, 68, 70, 72, 74, 76, 78, 80, 82, 84, 86, 88, 90, 92, 94, 96, 98, 100, 102, 104, 106, or any nucleotide sequence derived by degeneration of the genetic code of the abovementioned sequences and coding for the abovementioned mutated photoproteins.

The invention also relates to the vectors, in particular the plasmids, containing a recombinant sequence comprising a nucleotide sequence of the invention as defined above.

A subject of the invention is also the host cells, such as prokaryotic cells, in particular E. coli, or eukaryotic cells, in particular the HEK 293 (American Type Culture Collection ATCC No. CRL-1573) or CHO (ATCC No. CCL-61) lines, comprising a nucleotide sequence as defined above, said cells being as obtained by transformation using an abovementioned vector.

The invention also relates to any process for preparing mutated photoproteins as defined above, characterized in that it comprises the transformation of appropriate host cells using an abovementioned vector, the culture of transformed host cells thus obtained in an appropriate medium, and the recovery, if appropriate, after purification, of the mutated photoproteins produced by these cells, followed, if appropriate, by a stage of binding to the prosthetic group, such as coelenterazine.

A subject of the invention is also the use of mutated photoproteins as defined above, or of abovementioned transformed host cells expressing said mutated photoproteins, within the context of the implementation:
of processes for in vitro detection of molecules, such as proteins or antigens or nucleic acids in a biological sample, in particular within the context of in vitro screening for bacteria such as Listeria in food, or within the context of screening for pathogenic agents such as the HIV virus in humans,
of processes for detecting compounds with enzymatic activity in a biological sample, in particular within the context of screening for molecules activating or inhibiting a specific enzymatic activity,
of processes for detecting the intracellular calcium variations induced by various agents, in particular within the context of screening for molecules acting on a nucleic or protein sequence fused to the mutated photoprotein, or coexpressed with the mutated photoprotein in abovementioned host cells.

A more particular subject of the invention is the processes for in vitro detection of proteins or antigens or of nucleic acids in a biological sample, as defined above, characterized in that they principally comprise the following stages:
if appropriate, a stage of amplification of the number of nucleic acids present in the biological sample,
immobilization of the proteins or antigens or nucleic acids on an appropriate support, then the addition of a specific probe of said proteins or antigens or nucleic acids and rinsing, said probe being bound to a mutated photoprotein as defined above, or said photoprotein being added onto the support with the appropriate reagents for its binding to said probe,
measurement of the intensity of the bioluminescence emitted after the rinsing stage.

A subject of the invention is also the processes for in vitro detection of compounds with enzymatic activity in a biological sample as defined above, characterized in that they principally comprise the following stages:
immobilization on an appropriate support of a protein substrate specific to the enzymatic activity to be detected, this substrate being bound to a mutated photoprotein as defined above, addition of the biological sample, then rinsing,
or immobilization on an appropriate support of the compounds of the biological sample, addition of the protein substrate bound to a mutated photoprotein as defined above, then rinsing,
measurement of the intensity of the bioluminescence emitted after the rinsing stage.

A more particular subject of the invention is also, the processes for in vitro detection of the intracellular calcium variations induced by various agents as defined above, characterized in that they comprise the culture of abovementioned transformed cells, with the sample containing the molecules to be detected, and measurement of the variation in bioluminescence.

Advantageously, the abovementioned processes according to the invention, are characterized in that they can be carried out up to temperatures of approximately 50° C., using thermostable, and, if appropriate, persistent mutated photoproteins, as defined above.

Advantageously also, the abovementioned processes according to the invention, are characterized in that they can be carried out simultaneously on multiple samples, using persistent, and, if appropriate, thermostable photoproteins, as defined above.

A subject of the invention is also the kits for the implementation of processes as defined above, characterized in that they comprise the abovementioned mutated photoproteins, if appropriate in combination with reagents necessary for the implementation of said processes.

A more particular subject of the invention is the kits as defined above, characterized in that they can be kept in solutions which are ready for use, in particular for at least approximately 4 days at ambient temperatures of approximately 20° C., and being able to reach up to approximately 37° C., when they contain thermostable and, if appropriate, persistent mutated photoproteins as defined above.

The invention is further illustrated using the detailed description which follows, of the obtaining of mutated photoproteins as defined above, and of the conditions for using these photoproteins within the context of the abovementioned uses of the latter.

a) Obtaining Thermostable Mutants of the Photoprotein Aequorin

Protocols

The procedure used consisted of producing a bank of random mutants of aequorin generated by the “DNA shuffling” technique (Stemmer, WPC, 1994). These mutants, inserted in a prokaryotic expression vector were transformed in E. coli and cloned. The clones were screened individually for an increase in bioluminescence. The best mutants were then used for a second round of DNA shuffling followed by identical screening. This process was repeated a third time. Three mutations which increased the activity of aequorin were indexed. During the subsequent tests, we were able to show that these mutations do not increase the emission of light from aequorin but do increase its stability. This explains why these mutants were selected in our prokaryotic cell system (E. coli). For one of these mutants, the tests show an increase in the half-life time at 37° C. of a factor of 12.5 in the prokaryotic cell system and a factor of 7.5 for the purified protein, in comparison with the wild-type aequorin. Similarly, the semi-inactivation temperature of this mutant during a thermal shock of 30 minutes is 10 degrees Celsius higher than that of the wild-type aequorin (experiment carried out in cell system and on purified protein). This same mutant shows a slight reduction in affinity for calcium in comparison with the wild-type aequorin, which is an advantage for a use of aequorin in vitro.

DNA Shuffling

The cDNA of “wild-type” aequorin (Aeqwt, ˜600 bp) was sub-cloned at the sites KpnI (5′) and EcoRI (3′) of the vector pPD16 dependent on the Plac promoter. This clone was named pPD-Aeqwt. 20 ng of the plasmid pPD-Aeqwt was digested by PstI and EagI (sites external to the aequorin insert in the pPD16 polylinker) and the Aeqwt insert amplified by PCR using the following primers: up-e-Aeq, 5′CGG GTA CCG ATG CTTTATGATGTTCCTGAT 3′ and lo-e-Aeq, 5′ TGGAATTC TTA GGGGACAGCTCCAC 3′. The resultant PCR product was purified (Qiaquick extraction kit, Qiagen). 3 μg of the purified product was digested by DNAse I (1 ng/μl) in 100 μl of DNAse I buffer at 25° C. for 7 minutes. The digestion fragments comprised between 50 and 300 bp were purified by electrophoresis on agarose gel.

PCR without Primers (Shuffling)

1 μg of the digested Aeq fragments were subjected to a PCR without primers in 50 μl of PCR buffer containing 200 μM of each dNTP, 2.2 mM MgCl₂, 2.5 units of taq polymerase (Qiagen) by carrying out 35 cycles with 30 seconds at 94° C., 30 seconds at 45° C. and 30 seconds at 72° C.

PCR with Primers

2.5 μl of the shuffling reaction product was amplified by 20 PCR cycles (30 seconds at 94° C., 30 seconds at 58° C. and 40 seconds at 72° C.) in 100 μl of PCR buffer containing 20 pmole of each primer up-e-Aeq and lo-e-Aeq, 50 μM of each dNTP, 1.5 mM MgCl₂ and 2.5 units of taq polymerase (Qiagen).

Mutant Bank

The product of the PCR with primers (mutated aequorins) was purified (Qiaquick extraction kit, Qiagen), KpnI/EcoRI digested and sub-cloned in the pPD 16 vector dependent on the Plac promoter. The bank of mutant aequorins was transformed in the E. Coli strain XL1 blue (Stratagene), and plated on LB ampicillin dishes.

In the 1st round of shuffling-screening, 15840 colonies were sub-cultured individually, transferred to 96-well plates (Costar) in 50 μl of LB ampicillin per well and incubated for 4 hours at 37° C. with stirring with a view to screening their activity.

In the 2nd and 3rd rounds of shuffling-screening, respectively 19200 and 17952 colonies were sub-cultured individually, transferred to 96-well plates in 200 μl of freezing medium (composition in g/l, Bacto tryptone, 16, Bacto yeast extract, 10, NaCl, 5, K2HPO4, 0.27, KH2PO4, 7.16, Na citrate, 2, MgSO4-7H₂O, 0.1, (NH4)₂SO4, 0.9, glycerol, 50 and 100 μg/ml of ampicillin) and after incubation overnight at 37° C. without stirring, stored at −80° C. These storage plates were sub-cultured (96 pin replicator long, Genetix) in 96-well plates (Costar) in 50 μl of LB ampicillin per well. These sub-cultures for screening were incubated for 4 hours at 37° C. with stirring.

After the addition of 50 μl per well of a solution containing Tris pH8, 100 mM, NaCl, 90 mM, coelenterazine, 5 mM, the plates to be screened were incubated at 4° C. overnight for reconstitution of the aequorin (final volume per well 100 μl).

Screening

After 15 minutes at ambient temperature, the clones of mutated aequorins in 96 well plates were screened for their bioluminescence activity activated by Ca²⁺ using an injector luminometer (PhL, Mediators, Austria). The light emitted during the 4 seconds following the injection of 100 μl of a solution containing CaCl₂, 20 mM and triton X100, 1% was measured for each clone individually. The mutants were selected on the basis of an activity 15 times greater than the average of the clones from the 96 well plate and re-plated on LB ampicillin dishes for confirmation and comparison with the wild-type aequorin. At the end of the 1st round of screening, 40 clones, the activity of which was greater than that of the wild-type aequorin were selected for the 2nd round of shuffling-screening. 37 clones were selected in the second round for the third round of screening. The insert of each of these clones was amplified individually by PCR using the primers up-e-Aeq and lo-e-Aeq (see above, DNA shuffling). The PCR products of each clone were combined (200 ng per clone) and subjected to the previous DNA shuffling protocol in order to generate the bank of mutants of the following round.

Sequencing of the Mutants

The sequencing of the mutants (7 mutants selected in the 2nd round and 7 mutants selected in the 3rd round) was carried out on an ABI310 automatic sequencer (PE applied biosystems) with the primers up-e-Aeq and lo-e-Aeq (see DNA shuffling).

Purification of the Aequorins

The cDNAs of the wild-type aequorin and of the mutant aequorins, excised by KpnI/EcoRI double cleavage were sub-cloned in pRSETC (Invitrogen, Xpress protein expression system). The resultant plasmids were transformed in the E. Coli strain BL21(DE3) pLysS (Invitrogen) for expression of the aequorins. The aequorins were purified by affinity chromatography on a nickel-agarose column (Invitrogen, Xpress protein expression system), according to the manufacturer's instructions (elution with 350 mM imidazole). The purified aequorins were kept at −20° C. in a solution containing (final concentrations) imidazole, 175 mM; EDTA, 10 μM; BSA, 10 μg/ml and glycerol, 50%.

Expression in Eukaryotic Cell Line (HEK 293)

The cDNAs of the wild-type aequorin and of the mutant aequorins, excised by KpnI/SpeI or HindIII/SpeI double cleavage were sub-cloned at the KpnI/NheI or HindIII/NheI sites in a eucaryotic expression vector, pCMX, dependent on the CMV promoter. The resultant plasmids were co-transfected with a plasmid containing the LacZ gene (β-galactosidase) dependent on the RSV promoter in the HEK 293 cells. 24 hours after transfection the cells were collected and resuspended in PBS buffer.

β-galactosidase Activity Test.

An aliquot of the cell suspension was used to measure of the β-galactosidase activity (luminescent β-galactosidase detection kit II, Clontech) in 96-well plates in order to normalize the aequorin activities.

Aequorin Bioluminescence.

After the addition of coelenterazine (10 μM final) the cell suspension was distributed in 96-well plates at a rate of 50 μl per well and incubated for 3 hours at 37° C. in order to reconstitute the aequorin. The aequorin activity was measured using an injector luminometer (PhL, Mediators, Austria). The light emitted during the 4 seconds following the injection of 100 μl of a solution containing CaCl₂, 1.5 mM and triton X100, 0.75% was measured and normalized in comparison with the P-galactosidase activity.

Aequorin Stability Tests

In Bacteria

For each aequorin clone to be tested, a colony was amplified in 5 ml of LB ampicillin and after centrifugation the bacterial pellet was rinsed twice with 5 ml of a solution containing NaCl, 100 mM; Tris HCl, 50 mM, pH 8 and EGTA, 1 mM. The bacteria were resuspended in 500 μl of the same solution containing 10 μM of coelenterazine and incubated at 4° C. overnight. After the addition of lysozyme (0.8 mg/ml final) and homogenization, 50 μl aliquots were removed for the stability tests.

Stability at 37° C. over time: the aliquots were incubated at 37° C. and for different times (up to 72 hours), then were distributed in 96-well plates at a rate of 10 μl per well. The luminescence activated by the injection of 200 μl of a solution containing CaCl₂, 2 mM; NaCl, 100 mM; Tris HCl pH 8, 50 mM and EGTA, 1 mM (free Ca²⁺, ˜1 mM) was measured and normalized in comparison with the value obtained at to.

Stability at different temperatures: the aliquots were incubated at temperatures comprised between 25 and 55° C. for 30 minutes then distributed in 96-well plates at a rate of 10 μl per well. The luminescence, measured as above, was normalized in comparison with the value obtained at 25° C.

On Purified Proteins

The purified aequorins were reconstituted by 10-fold dilution in a solution containing Tris HCl, 50 mM pH8; DTT, 10 mM, EDTA, 1 mM and coelenterazine, 2 μM and incubation for 1 hour at 4° C. 50 μl aliquots were then removed for the stability test. The stability measurements were carried out as previously, with the exception of the activation stage carried out by the injection of 100 μl of a solution containing CaCl₂, 10 mM; Tris HCl pH 8, 50 mM and EDTA, 1 mM.

Calcium Sensitivity Tests of Aequorins

The purified aequorins were reconstituted by 10-fold dilution in a solution containing Tris HCl, 50 mM pH8; DTT, 10 mM, EDTA, 10 μM and coelenterazine, 2 μM and incubation for 1 hour at 4° C. The reconstituted aequorins were distributed in 96-well plates at a rate of 55 μl per well and activated by 100 μl of a solution containing Tris HCl pH 8, 50 mM; EDTA, 10 μM and variable concentrations of free Ca²⁺ (final free concentrations after injection: 10⁻⁸ to 10⁻¹ M). The luminescence measurements (PhL luminometer, Mediators) were carried out in kinetic mode (Fast kinetics, Interval 0.1 second, kinetic points 60). The determination of the calcium sensitivity curves was based on the initial luminescence values of the light emission kinetics.

B) Obtaining Persistent Mutants of the Photoprotein Aequorin with Prolonged Luminescence

Protocols

The procedure used consisted of producing a bank of random mutants of aequorin generated by the “DNA shuffling” technique (Stemmer, WPC, 1994). These mutants, inserted in a prokaryotic expression vector were transformed in E. Coli and cloned. The clones were screened individually for an increase in the duration of bioluminescence emission. The best mutants were then sequenced. We indexed six mutations which prolong the bioluminescence of the aequorin. During subsequent tests, we were able to show that these mutations do not increase the total light emission of the aequorin but slow down its kinetics. For these mutants, the tests show an increase in bioluminescence emission time (of the order of a minute) by approximately a factor of ten in prokaryotic or eukaryotic cell systems and on purified protein, in comparison with the wild-type aequorin (of the order of a second). Preliminary data indicate that certain mutants would have a thermostability greater than that of the wild-type aequorin (experiments carried out on purified protein). All these mutants except one show a considerable reduction in affinity for calcium in comparison with the wild-type aequorin.

DNA Shuffling

The cDNA of the “wild type” aequorin (Aeqwt, ˜600 bp) was sub-cloned at the sites KpnI (5′) and EcoRI (3′) of the vector pPD16 dependent on the Plac promoter. This clone was named pPD-Aeqwt. 20 ng of the plasmid pPD-Aeqwt was digested by PstI and EagI (sites external to the aequorin insert in the pPDl6 polylinker) and the Aeqwt insert amplified by PCR using the following primers: up-e-Aeq, 5′CGG GTA CCG ATG CTTTATGATGTTCCTGAT 3′ and lo-e-Aeq, 5′ TGGAATTC TTA GGGGACAGCTCCAC 3′. The resultant PCR product was purified (Qiaquick extraction kit, Qiagen). 3 μg of the purified product was digested by DNAse I (1 ng/μl) in 100 μl of DNAse I buffer at 25° C. for 7 minutes. The digestion fragments comprised between 50 and 300 bp were purified by electrophoresis on agarose gel.

PCR without Primers (Shuffling)

1 μg of the digested Aeq fragments were subjected to PCR without primers in 50 μl of PCR buffer containing 200 μM of each dNTP, 2.2 mM MgCl₂, 2.5 units of taq polymerase (Qiagen) by carrying out 35 cycles with 30 seconds at 94° C., 30 seconds at 45° C. and 30 seconds at 72° C.

PCR with Primers

2.5 μl of the shuffling reaction product was amplified by 20 PCR cycles (30 seconds at 94° C., 30 seconds at 58° C. and 40 seconds at 72° C.) in 100 μl of PCR buffer containing 20 pmole each of primer up-e-Aeq and lo-e-Aeq, 50 μM each of dNTP, 1.5 mM MgCl₂ and 2.5 units of taq polymerase (Qiagen).

Mutant Bank

The product of the PCR with primers (mutated aequorins) was purified (Qiaquick extraction kit, Qiagen), KpnI/EcoRI digested and sub-cloned in the pPD16 vector dependent on the Plac promoter. The bank of mutant aequorins was transformed in the E. Coli strain XL1 blue (Stratagene), and plated on LB ampicillin dishes.

15840 colonies were sub-cultured individually, transferred to 96-well plates (Costar) in 50 μl of LB ampicillin per well and incubated for 4 hours at 37° C. with stirring with a view to screening their activity.

After the addition of 50 μl per well of a solution containing Tris pH8, 100 mM, NaCl, 90 mM, coelenterazine, 5 mM, the plates to be screened were incubated at 4° C. overnight for the reconstitution of the aequorin (final volume per well 100 μl).

Screening

After 15 minutes at ambient temperature, the clones of mutated aequorins in 96-well plates were screened for their bioluminescence activity activated by the Ca²⁺ using an injector luminometer (PhL, Mediators, Austria). The light emitted during the 4 seconds (t0-4) following the injection of 100 μl of a solution containing CaCl₂, 20 mM and triton X100, 1%, and during the 4 following seconds (t4-8) was measured for each clone individually. The mutants having a t0-4/t4-8 ratio of less than 1.5 were selected and re-plated on LB ampicillin dishes for confirmation and determination of the best mutants.

Sequencing of the Mutants

The sequencing of the mutants (20 mutants sequenced) was carried out on an ABI310 automatic sequencer (PE applied biosystems) with the primers up-e-Aeq and lo-e-Aeq (see DNA shuffling).

Purification of the Aequorins

The cDNAs of the wild-type aequorin and of the mutant aequorins, excised by KpnI/EcoRI double cleavage were sub-cloned in pRSETC (Invitrogen, Xpress protein expression system). The resultant plasmids were transformed in the E. Coli strain BL21(DE3) pLysS (Invitrogen) for expression of the aequorins. The aequorins were purified by affinity chromatography on a nickel-agarose column (Invitrogen, Xpress protein expression system), according to the manufacturer's instructions (elution with 350 mM imidazole). The purified aequorins were kept at −20° C. in a solution containing (final concentrations) imidazole, 175 mM; EDTA, 10 μM; BSA, 10 μg/ml and glycerol, 50%.

Expression in Eukaryotic Cell Line (HEK 293)

The cDNAs of the wild-type aequorin and of the mutant aequorins, excised by KpnI/SpeI or HindIII/SpeI double cleavage were sub-cloned at the KpnI/Nhel or HindIII/NheI sites in a eucaryotic expression vector, pCMX, dependent on the CMV promoter. The resultant plasmids were transfected in the HEK 293 cells. 24 hours after transfection, the cells were collected and resuspended in PBS buffer.

After the addition of coelenterazine (10 μM final) the cell suspension was distributed in 96-well plates at a rate of 50 μl per well and incubated for 3 hours at 37° C. in order to reconstitute the aequorin. The aequorin activity was measured using an injector luminometer (PhL, Mediators, Austria). The bioluminescence emission kinetics following the injection of 100 μl of a solution containing CaCl₂, 1.5 mM and triton X100, 0.75% were determined in kinetic mode (Fast kinetics, Interval 0,1-10 seconds, kinetic points 60).

Bioluminescence Kinetics and Calcium Sensitivity Tests of Aequorins

In Bacteria

For each aequorin clone to be tested, a colony was amplified in 5 ml of LB ampicillin and after centrifugation the bacterial pellet was rinsed twice with 5 ml of a solution containing NaCl, 100 mM; Tris HCl, 50 mM, pH 8 and EGTA, 1 mM. The bacteria were resuspended in 500 μl of the same solution containing 10 μM of coelenterazine and incubated at 4° C. overnight. After the addition of lysozyme (0.8 mg/ml final) and homogenization, 50 μl aliquots were removed for the kinetics and/or calcium sensitivity test as described below.

On Purified Proteins

The purified aequorins were reconstituted by 10-fold dilution in a solution containing Tris HCl, 50 mM pH8; DTT, 10 mM, EDTA, 10 μM and coelenterazine, 2 μM and incubation for 1 hour at 4° C. The reconstituted aequorins were distributed in 96-well plates at a rate of 55 μl per well and activated by 100 μl of a solution containing Tris HCl pH 8, 50 mM; EDTA, 10 μM and variable concentrations of free Ca²⁺ (final free concentrations after injection: 10⁻⁸ to 10⁻¹ M). The luminescence measurements (luminometer PhL, Mediators) were carried out in kinetic mode (Fast kinetics, Interval 0,1-10 seconds, kinetic points 60). The determination of the calcium sensitivity curves was based on the initial luminescence values of the light emission kinetics.

C) Industrial Uses of the Mutants of the Photoprotein Aequorin

1) In Vitro Detection of Organic Molecules (Nucleic Acids, Proteins, Antigens Etc.)

These detection tests are based on the immobilization of the molecule to be detected and on the specific binding of the photoprotein to this molecule. The immobilization and the specific binding are carried out by widely varying means as a function of the type of molecule to be detected and the type of sample to be analyzed. The quantity of the molecule to be detected in the sample is then determined by activation of the bound photoprotein and measurement of the bioluminescence emitted.

a) Detection of Nucleic Acids

The detection of nucleic acid sequences can be carried out, either after an amplification stage by PCR (DNA) or RT-PCR(RNA) or any other nucleic acid amplification technique, or directly. After immobilization of the nucleic acid molecules or their amplification products, the molecules are detected by hybridization of a probe. The hybridization of the probe is then revealed thanks to a photoprotein coupled directly to the probe or subsequently bound to the latter. The nucleic acid molecules or their amplification products are quantified by the intensity of the bioluminescence emitted.

The principal bibliographical references describing such processes are the following:
1—Lewis J C, Daunert S. Photoproteins as luminescent labels in binding assays. Fresenius J Anal Chem. 2000 Mar-Apr; 366(6-7):760-8
2—Coombes BK, Mahony JB. Nucleic acid sequence based amplification (NASBA) of Chiamydia pneumoniae major outer membrane protein (ompA) mRNA with bioluminescent detection. Comb Chem High Throughput Screen. 2000 August;3(4):315-27.
3—Laios E, Ioannou P C, Christopoulos T K. Enzyme-amplified aequorin-based bioluminometric hybridization assays. Anal Chem. 2001 Feb. 1; 73(3):689-92.
4—Actor J K. Bioluminescent quantitation and detection of gene expression during infectious disease. Comb Chem High Throughput Screen. 2000 August; 3(4):273-88.
5—White S R, Christopoulos TK. Signal amplification system for DNA hybridization assays based on in vitro expression of a DNA label encoding apoaequorin. Nucleic Acids Res. 1999 Oct. 1; 27(19):e25.
6—Guenthner P C, Hart THIS. Quantitative, competitive PCR assay for HIV-1 using a microplate-based detection system. Biotechnic. 1998 May;24(5):810-6.

b) Detection of Proteins or Antigens

After immobilization, the proteins and antigens are detected by specific combination with a probe (combination of the antigen-antibody or ligand-receptor type). The binding of the probe is then revealed thanks to a photoprotein coupled directly to the probe or subsequently bound to the latter. The protein or antigen molecules are quantified by the intensity of the bioluminescence emitted.

The principal bibliographical references describing such processes are the following:
1-Jackson R J, Fujihashi K, Kiyono H, McGhee J R. Luminometry: a novel bioluminescent immunoassay enhances the quantitation of mucosal and systemic antibody responses. J Immunol Methods. 1996 Apr. 19;190(2):189-97.
2-Mattox S, Walrath K, Ceiler D, Smith DF, Cummings RD. A solid-phase assay for the activity of CMPNeuAc:Gal beta 1-4GlcNAc-R alpha-2,6-sialyltransferase. Anal Biochem. 1992 Nov. 1;206(2):430-6.

2) Detection of Enzymatic Activities

Protein substrates composed partly of a photoprotein (fusion protein type) can make it possible to measure enzymatic activities in vitro or in cell systems. The detectable enzymatic activities are, for example, of the “protein-modification” type (proteases, kinases, glycosylases, etc.). This type of measurement can serve for the screening of molecules activating or inhibiting a specific enzymatic activity (for example in reference 1, detection of HIV-1 protease activity).

For in vitro measurements, the protein substrate containing the photoprotein, or the sample to be assayed, are immobilized. The enzymatic activity is then quantified by the intensity of the bioluminescence emitted. The same type of measurement can be carried out in cell systems expressing the gene corresponding to the protein substrate containing the photoprotein. The cellular enzymatic activities are then quantified by activation of the photoprotein and measurement of the intensity of the bioluminescence emitted.

Such a process is described in particular in Deo S K, Lewis J C, Daunert S. Bioluminescence detection of proteolytic bond cleavage by using recombinant aequorin. Anal Biochem. 2000 May 15;281(1):87-94.

3) Detection Of Intracellular Calcium Variations in Cell Systems.

The aequorin type photoproteins expressed in (prokaryotic or eukaryotic) cell lines make it possible to detect the intracellular calcium variations induced by various agents. The intracellular calcium variations are detected by the corresponding variations in the bioluminescence emitted. The most common application uses HEK 293 type eukaryotic lines co-expressing aequorin and a neurotransmitter receptor (receptor-channel or G-protein-coupled receptor) for screening pharmacological agents or natural ligands acting on the receptor. Conversely, these systems can be used for screening DNA banks in the search for receptors activated by a pharmacological agent or natural ligand. The screening is based on the variation in bioluminescence induced by the application of the pharmacological agent or natural ligand.

The principal bibliographical references describing such processes are the following:
1—Button D, Brownstein M. Aequorin-expressing mammalian cell lines used to report Ca2+ mobilization. Cell Calcium. 1993 October;14(9):663-71.
2—Ungrin M D, Singh L M, Stocco R, Sas O F, Abramovitz M. An automated aequorin luminescence-based functional calcium assay for G-protein-coupled receptors. Anal Biochem. 1999 Jul. 15;272(1):34-42.
3—Schaeffer M T, Cully D, Chou M, Liu J, Van der Ploeg L H, Fong T M. Use of bioluminescent aequorin for the pharmacological characterization of 5HT receptors. J Recept Signal Transduct Res. 1999 November;19(6):927-38.
4—George S E, Schaeffer M T, Cully D, Beer M S, McAllister G. A high-throughput glow-type aequorin assay for measuring receptor-mediated changes in intracellular calcium levels. Anal Biochem. 2000 Nov. 15;286(2):231-7.
5—Parnot C, Bardin S, Miserey-Lenkei S, Guedin D, Corvol P, Clauser E. Systematic identification of mutations that constitutively activate the angiotensin II type 1A receptor by screening a randomly mutated cDNA library with an original pharmacological bioassay. Proc Natl Acad Sci USA. 2000 Jun. 20;97(13):7615-20.
6—Kotani M, Mollereau C, Detheux M, The Poul E, Brezillon S, Vakili J, Mazarguil H, Vassart G, Zajac J M, Parmentier M. Functional characterization of a human receptor for neuropeptide FF and related peptides. Br J. Pharmacol. 2001 May 1;133(1):138-144.

1. Use of photoproteins isolated from jellyfish for the preparation of mutated photoproteins having a thermostability greater than that of the photoproteins from which they derive, also designated thermostable mutated photoproteins, and/or by a luminescence time greater than that of the photoproteins from which they derive, also designated persistent mutated photoproteins, said mutated photoproteins being characterized in that their stability over time is increased at 37° C. by a factor of at least approximately 10, and/or in that their luminescence time is increased by a factor of at least approximately 10, in comparison with the photoproteins from which they derive.

2. Process for preparing thermostable and/or persistent mutated photoproteins, said mutated photoproteins being characterized in that their stability over time is increased at 37° C. by a factor of at least approximately 10, and/or in that their luminescence time is increased by a factor of at least approximately 10, in comparison with the jellyfish photoproteins from which they derive, characterized in that it comprises the implementation of one or more mutations of said jellyfish photoproteins, said mutations being chosen from:
at least one of the three mutations increasing the thermostability of said photoproteins, and chosen from the following:
suppression of the lysine (K) contained in the unit RHKX₁MFX₂ in which X₁=H or F, and X₂=N or D, this peptide unit being preserved in said photoproteins, or substitution of this lysine by a natural or non-natural amino acid, in particular substitution of this lysine by an arginine,
suppression of the glutamine (Q) contained in the unit DEMTRQHLGFWY, this peptide unit being preserved in said photoproteins, or substitution of this glutamine by a natural or non-natural amino acid, in particular substitution of this glutamine by an arginine,
suppression of the leucine (L) contained in the abovementioned unit DEMTRQHLGFWY, or substitution of this leucine by a natural or non-natural amino acid, in particular substitution of this leucine by an isoleucine,
and/or at least one of the six mutations increasing the luminescence time of said photoproteins, and chosen from the following:
suppression of the glutamate (E) contained in the unit DX₁NX₂X₃GX₄IX₅LX₆E in which X₁=V or I, X₂=H, G or S, X₃=N or D, X₄=K or Q, X₅=S, T or N, and X₆=D or N, this peptide unit being preserved in said photoproteins, or substitution of this glutamate by a natural or non-natural amino acid, in particular substitution of this glutamate by a small amino acid such as glycine, alanine, cysteine, or threonine,
suppression of the first aspartate (D) contained in the unit DKDX₁X₂GX₃X₄X₅LDE in which X₁=Q, G or R, X₂=N or S, X₃=A, S or T, X₄=I or V, and X₅=T or S, this peptide unit being preserved in said photoproteins, or substitution of this aspartate by a natural or non-natural amino acid, in particular substitution of this aspartate by a small amino acid such as glycine, alanine, cysteine, or threonine,
suppression of the glutamate (E) contained in the abovementioned unit DKDX₁X₂GX₃X₄X₅LDE, this peptide unit being preserved in said photoproteins, or substitution of this glutamate by a natural or non-natural amino acid, in particular substitution of this glutamate by a small amino acid such as glycine, alanine, cysteine, or threonine,
suppression of the phenylalanine (F) contained in the unit EX₁TFX₂X₃ in which X₁=E, K or A, X₂=R, K or A, and X₃=V or H, this peptide unit being preserved in said photoproteins, or substitution of this phenylalanine by a natural or non-natural amino acid, in particular substitution of this phenylalanine by a serine, or by a small amino acid such as glycine, alanine, cysteine, or threonine,
suppression of the first aspartate (D) contained in the unit DX₁DX₂X₃GX₄LDVDE, in which X₁=I or L, X₂=E, N or G, X₃=S or D, and X₄=Q, K or D, this peptide unit being preserved in said photoproteins, or substitution of this aspartate by a natural or non-natural amino acid, in particular substitution of this aspartate by a small amino acid such as glycine, alanine, cysteine, or threonine,
suppression of the valine in position 54 of the peptide sequence SEQ ID NO: 2 of aequorin, or substitution of this valine by a natural or non-natural amino acid, in particular substitution of this valine by an alanine, or by a small amino acid such as glycine, alanine, cysteine, or threonine.

3. Mutated photoproteins derived from the photoproteins isolated from jellyfish, characterized in that their stability over time is increased at 37° C. by a factor of at least approximately 10, and/or in that their luminescence time is increased by a factor of at least approximately 10, in comparison with the photoproteins from which they derive.

4. Mutated photoproteins according to claim 3, characterized in that they are stable for at least approximately 30 minutes up to a temperature of approximately 50° C., and in that they can be kept for at least approximately 4 days at temperatures which can reach up to 37° C., and/or in that their luminescence time is comprised between approximately 1 minute and approximately 5 minutes.

5. Mutated photoproteins according to claim 3, characterized in that they comprise
at least one of the three mutations increasing their thermostability, chosen from the following:
suppression of the lysine (K) contained in the unit RHKX₁MFX₂ in which X₁=H or F, and X₂=N or D, this peptide unit being preserved in said photoproteins, or substitution of this lysine by a natural or non-natural amino acid, in particular substitution of this lysine by an arginine,
suppression of the glutamine (Q) contained in the unit DEMTRQHLGFWY, this peptide unit being preserved in said photoproteins, or substitution of this glutamine by a natural or non-natural amino acid, in particular substitution of this glutamine by an arginine,
suppression of the leucine (L) contained in the abovementioned unit DEMTRQHLGFWY, or substitution of this leucine by a natural or non-natural amino acid, in particular substitution of this leucine by an isoleucine,
and/or at least one of the six mutations increasing their luminescence time, chosen from the following:
suppression of the glutamate (E) contained in the unit DX₁NX₂X₃GX₄IX₅LX₆E in which X₁=V or I, X₂=H, G or S, X₃=N or D, X₄=K or Q, X₅=S, T or N, and X₆=D or N, this peptide unit being preserved in said photoproteins, or substitution of this glutamate by a natural or non-natural amino acid, excluding serine, in particular substitution of this glutamate by a small amino acid such as glycine, alanine, cysteine, or threonine,
suppression of the first aspartate (D) contained in the unit DKDX₁X₂GX₃X₄X₅LDE in which X₁=Q, G or R, X₂=N or S, X₃=A, S or T, X₄=I or V, and X₅=T or S, this peptide unit being preserved in said photoproteins, or substitution of this aspartate by a natural or non-natural amino acid, excluding serine, in particular substitution of this aspartate by a small amino acid such as glycine, alanine, cysteine, or threonine,
suppression of the glutamate (E) contained in the abovementioned unit DKDX₁X₂GX₃X₄X₅LDE, this peptide unit being preserved in said photoproteins, or substitution of this glutamate by a natural or non-natural amino acid, excluding serine, in particular substitution of this glutamate by a small amino acid such as glycine, alanine, cysteine, or threonine,
suppression of the phenylalanine (F) contained in the unit EX₁TFX₂X₃ in which X₁=E, K or A, X₂=R, K or A, and X₃=V or H, this peptide unit being preserved in said photoproteins, or substitution of this phenylalanine by a natural or non-natural amino acid, in particular substitution of this phenylalanine by a serine, or by a small amino acid such as glycine, alanine, cysteine, or threonine,
suppression of the first aspartate (D) contained in the unit DX₁DX₂X₃GX₄LDVDE, in which X₁=I or L, X₂=E, N or G, X₃=S or D, and X₄=Q, K or D, this peptide unit being preserved in said photoproteins, or substitution of this aspartate by a natural or non-natural amino acid, excluding serine, in particular substitution of this aspartate by a small amino acid such as glycine, alanine, cysteine, or threonine,
suppression of the valine in position 54 of the peptide sequence SEQ ID NO: 2 of aequorin, or substitution of this valine by a natural or non-natural amino acid, in particular substitution of this valine by an alanine, or by a small amino acid such as glycine, alanine, cysteine, or threonine.

6. Thermostable mutated photoproteins according to claim 3, characterized in that they include at least one of the following two mutations:
suppression of the glutamine contained in the unit DEMTRQHLGFWY, this peptide unit being preserved in said photoproteins, or substitution of this glutamine by a natural or non-natural amino acid, in particular substitution of this glutamine by an arginine,
suppression of the leucine contained in the abovementioned unit DEMTRQHLGFWY, or substitution of this leucine by a natural or non-natural amino acid, in particular substitution of this leucine by an isoleucine.

7. Persistent mutated photoproteins according to claim 3, characterized in that they comprise at least one of the following mutations:
suppression of the first aspartate (D) contained in the unit DKDX₁X₂GX₃X₄X₅LDE in which X₁=Q, G or R, X₂=N or S, X₃=A, S or T, X₄=I or V, and X₅=T or S, this peptide unit being preserved in said photoproteins, or substitution of this aspartate by a natural or non-natural amino acid, excluding serine, in particular substitution of this aspartate by a small amino acid such as glycine, alanine, cysteine, or threonine,
suppression of the glutamate (E) contained in the abovementioned unit DKDX₁X₂GX₃X₄X₅LDE, this peptide unit being preserved in said photoproteins, or substitution of this glutamate by a natural or non-natural amino acid, excluding serine, in particular substitution of this glutamate by a small amino acid such as glycine, alanine, cysteine, or threonine,
suppression of the first aspartate (D) contained in the unit DX₁DX₂X₃GX₄LDVDE, in which X₁=I or L, X₂=E, N or G, X₃=S or D, and X₄=Q, K or D, this peptide unit being preserved in said photoproteins, or substitution of this aspartate by a natural or non-natural amino acid, excluding serine, in particular substitution of this aspartate by a small amino acid such as glycine, alanine, cysteine, or threonine.

8. Mutated photoproteins according to claim 3, characterized in that they derive from aequorin (SEQ ID NO: 2), clytin (SEQ ID NO: 18), mitrocomin (SEQ ID NO: 34), and obelin (SEQ ID NO: 50).

9. Thermostable mutated photoproteins according to claim 8, chosen from:
the proteins derived from aequorin comprising the following sequences: SEQ ID NO: 4, 6, 8, 10, 12, 14, 16,
the proteins derived from clytin comprising the following sequences: SEQ ID NO: 20, 22, 24, 26, 28, 30, 32,
the proteins of mitrocomin comprising the following sequences: SEQ ID NO: 36, 38, 40, 42, 44, 46, 48,
the proteins derived from obelin comprising the following sequences: SEQ ID NO: 52, 54, 56, 58, 60, 62, 64.

10. Persistent mutated photoproteins according to claim 8, chosen from:
the proteins derived from aequorin comprising the following sequences: SEQ ID NO: 66, 68, 70, 72, 74, 76,
the proteins derived from clytin comprising the following sequences: SEQ ID NO: 78, 80, 82, 84, and 86,
the proteins of mitrocomin comprising the following sequences: SEQ ID NO: 88, 90, 92, 94, and 96,
the proteins derived from obelin comprising the following sequences: SEQ ID NO: 98, 100, 102, 104, and 106.

11. Thermostable and persistent mutated photoproteins according to claim 8, chosen from:
the proteins derived from aequorin of sequences SEQ ID NO: 107, 108, 109, 110, 111, 112, 113, and corresponding to the sequences SEQ ID NO: 4, 6, 8, 10, 12, 14, 16, in which E in position 45 is replaced by G, and/or V in position 54 is replaced by A, and/or D in position 127 is replaced by G, and/or E in position 138 is replaced by G, and/or F in position 159 is replaced by S, and/or D in position 163 is replaced by G,
the proteins derived from clytin of sequences SEQ ID NO: 114, 115, 116, 117, 118, 119, 120, and corresponding to the following sequences: SEQ ID NO: 20, 22, 24, 26, 28, 30, 32, in which E in position 44 is replaced by G, and/or D in position 126 is replaced by G, and/or E in position 137 is replaced by G, and/or F in position 158 is replaced by S, and/or D in position 162 is replaced by G,
the proteins of mitrocomin of sequences SEQ ID NO: 121, 122, 123, 124, 125, 126, 127, and corresponding to the following sequences: SEQ ID NO: 36, 38, 40, 42, 44, 46, 48, in which E in position 43 is replaced by G, and/or D in position 125 is replaced by G, and/or E in position 136 is replaced by G, and/or F in position 157 is replaced by S, and/or D in position 161 is replaced by G,
the proteins derived from obelin of sequences SEQ ID NO: 128, 129, 130, 131, 132, 133, 134, and corresponding to the following sequences: SEQ ID NO: 52, 54, 56, 58, 60, 62, 64, in which E in position 41 is replaced by G, and/or D in position 123 is replaced by G, and/or E in position 134 is replaced by G, and/or F in position 155 is replaced by S, and/or D in position 159 is replaced by G.

12. Mutated photoproteins according to claim 3, characterized in that they are bound:
to a protein or nucleic probe capable of recognizing specific antigens or proteins or nucleic acids,
or to a specific substrate with a specific enzymatic activity,
or to a molecule capable of forming a complex with another molecule, such as the avidin-biotin complex.

13. Nucleotide sequences coding for the mutated photoproteins according to claim 3.

14. Nucleotide sequences, coding for the mutated photoproteins, chosen from the nucleic acids comprising the sequences SEQ ID NO: 3, 5, 7, 9, 11, 13, 15, 19, 21, 23, 25, 27, 29, 31, 35, 37, 39, 41, 43, 45, 47, 51, 53, 55, 57, 59, 61, 63, 65, 67, 69, 71, 73, 75, 77, 79, 81, 83, 85, 87, 89, 91, 93, 95, 97, 99, 101, 103, 105, or any nucleotide sequence derived by degeneration of the genetic code of the abovementioned sequences and coding for the mutated photoproteins.

15. Vector, such as a plasmid, containing a recombinant sequence comprising a nucleotide sequence according to claim 13.

16. Host cells, such as prokaryotic cells, in particular E. coli, or eukaryotic cells, in particular the HEK 293, or CHO lines, transformed by a vector according to claim 15.

17. Process for preparing mutated photoproteins according to claim 3, characterized in that it comprises the use of a vector capable of producing said photoproteins, or the transformation of appropriate host cells using an abovementioned vector, the culture of host cells transformed thus obtained in an appropriate medium, and the recovery, if appropriate after purification, of the mutated photoproteins produced by these cells.

18. Use of mutated photoproteins according to claim 3, within the context of the implementation:
of processes for in vitro detection of molecules, such as proteins or antigens or nucleic acids in a biological sample, in particular within the context of in vitro screening for bacteria such as Listeria in food, or within the context of screening for pathogenic agents such as the HIV virus in humans,
of processes for detecting compounds with enzymatic activity in a biological sample, in particular within the context of screening for molecules activating or inhibiting a specific enzymatic activity,
of processes for detecting intracellular calcium variations induced by various agents, in particular within the context of screening for molecules acting on a nucleic or protein sequence fused to the mutated photoprotein, or coexpressed with the mutated photoprotein in above-mentioned host cells.

19. Processes for in vitro detection of proteins or antigens or of nucleic acids in a biological sample, as defined in claim 18, characterized in that they principally comprise the following stages:
if appropriate, a stage of amplification of the number of nucleic acids present in the biological sample,
immobilization of the proteins or antigens or nucleic acids on an appropriate support, then the addition of a specific probe of said proteins or antigens or nucleic acids and rinsing, said probe being bound to a photoprotein, or said photoprotein being added to the support with the appropriate reagents for its binding to said probe,
measurement of the intensity of the bioluminescence emitted after the rinsing stage.

20. Processes for in vitro detection of compounds with enzymatic activity in a biological sample as defined in claim 18, characterized in that they principally comprise the following stages:
immobilization on an appropriate support of a protein substrate specific to the enzymatic activity to be detected, this substrate being bound to a photoprotein according to claim 12, addition of the biological sample, then rinsing,
or immobilization on an appropriate support of the compounds of the biological sample, addition of the protein substrate bound to a photoprotein according to claim 12, then rinsing,
measurement of the intensity of the bioluminescence emitted after the rinsing stage.

21. Processes for in vitro detection of the intracellular calcium variations induced by various agents as defined in claim 18, characterized in that it comprises the culture of cells transformed, with the sample containing the molecules to be detected, and measurement of the variation in bioluminescence.

22. Processes according to claim 19, characterized in that they can be carried out up to temperatures of approximately 50° C., using thermostable and, if appropriate, persistent photoproteins.

23. Processes according to claim 19, characterized in that they can be carried out simultaneously on multiple samples, using persistent and, if appropriate, thermostable photoproteins.

24. Kits for the implementation of processes according to claim 19, characterized in that they comprise mutated photoproteins, if appropriate in combination with reagents necessary for the implementation of said processes.

25. Kits according to claim 24, characterized in that they can be kept in solutions ready for use, in particular for at least 4 days at ambient temperatures of approximately 20° C. and being able to reach approximately 37° C., when they contain thermostable and, if appropriate, persistent photoproteins.
